SEQUENCE LISTING (42 sequences)

SEQ ID NO: 1 - DNA, 24 bp, Artificial Sequence
  Synthetic oligonucleotide
gccaagctagtcgactctagagga

SEQ ID NO: 2 - DNA, 28 bp, Artificial Sequence
  Synthetic oligonucleotide
gccaagctagtcgatcgactctagagga

SEQ ID NO: 3 - DNA, 28 bp, Artificial Sequence
  Synthetic oligonucleotide
gccaagctagtcgatcgactctagagga

SEQ ID NO: 4 - DNA, 24 bp, Artificial Sequence
  Synthetic oligonucleotide
gccaagctagtcgactctagagga

SEQ ID NO: 5 - DNA, 24 bp, Artificial Sequence
  Synthetic oligonucleotide
gccaagctagtcgactctagagga

SEQ ID NO: 6 - DNA, 24 bp, Artificial Sequence
  Synthetic oligonucleotide
gccaagctagtcgactctagagga

SEQ ID NO: 7 - DNA, 24 bp, Artificial Sequence
  Synthetic oligonucleotide
gccaagctagtcgactctagagga

SEQ ID NO: 8 - DNA, 21 bp, Artificial Sequence
  Synthetic oligonucleotide
gccaagctcgactctagagga

SEQ ID NO: 9 - DNA, 24 bp, Artificial Sequence
  Synthetic oligonucleotide
gccaagctagtcgactctagagga

SEQ ID NO: 10 - DNA, 28 bp, Artificial Sequence
  Synthetic oligonucleotide
gccaagctagtcgatcgactctagagga

SEQ ID NO: 11 - DNA, 26 bp, Artificial Sequence
  Synthetic oligonucleotide
gccaagctagtcgagactctagagga

SEQ ID NO: 12 - DNA, 26 bp, Artificial Sequence
  Synthetic oligonucleotide
gccaagctagtctcgactctagagga

SEQ ID NO: 13 - DNA, 49 bp, Artificial Sequence
  Synthetic oligonucleotide JS001
tagcaagcttgctagcggccgctcgaggccggcaaggccggatccagac

SEQ ID NO: 14 - DNA, 49 bp, Artificial Sequence
  Synthetic oligonucleotide JS002
gtctggatccggccttgccggcctcgagcggccgctagcaagcttgcta

SEQ ID NO: 15 - DNA, 43 bp, Artificial Sequence
  Synthetic oligonucleotide JS011
gcacgtcaggcacggcgtcctgctagcaagcttgctagcggcc

SEQ ID NO: 16 - DNA, 43 bp, Artificial Sequence
  Synthetic oligonucleotide JS025
gcacgtcaggcacggcgtctacgggactttcctacttggcagt

SEQ ID NO: 17 - DNA, 42 bp, Artificial Sequence
  Synthetic oligonucleotide JS034
gcacgtcaggcacggcgtcacttactctagcttcccgcaaca

SEQ ID NO: 18 - DNA, 43 bp, Artificial Sequence
  Synthetic oligonucleotide JS044
gcacgtcaggcacggcgtctgaagatcagttgggtgcacgagt

SEQ ID NO: 19 - DNA, 43 bp, Artificial Sequence
  Synthetic oligonucleotide JS065
gcacgtcaggcacggcgtcacattcaaccgattgagggaggga

SEQ ID NO: 20 - DNA, 120 bp, Artificial Sequence
  Synthetic oligonucleotide JS122
gcacgtcaggcacggcgtcagctgggtaccagctgctagcaagcttgctagcggccgctcgaggccggcaaggccggatccagacatgataagatacattggccactggacaccgtgttg

SEQ ID NO: 21 - DNA, 120 bp, Artificial Sequence
  Synthetic oligonucleotide G=O template
gcacgtcaggcacggcgtcagctgggtaccagctgctagcaagcttgctagcggccgctcnaggccggcaaggccggatccagacatgataagatacattggccactggacaccgtgttg

SEQ ID NO: 22 - DNA, 60 bp, Artificial Sequence
  Synthetic oligonucleotide JS124
gtcaggttcaccgatggagcgcaatgtatcttatcatgtctggatccggccttgccggcc

SEQ ID NO: 23 - DNA, 61 bp, Artificial Sequence
  Synthetic oligonucleotide JS125
gtcaggttcaccgatggagcgcaatgtatcttatcatgtctggatccggccttgccggcct

SEQ ID NO: 24 - DNA, 20 bp, Artificial Sequence
  Synthetic oligonucleotide JS003
tagcaagcttgctagcggcc

SEQ ID NO: 25 - DNA, 19 bp, Artificial Sequence
  Synthetic oligonucleotide JS004
gtctggatccggccttgcc

SEQ ID NO: 26 - DNA, 19 bp, Artificial Sequence
  Synthetic oligonucleotide JS012
gcacgtcaggcacggcgtc

SEQ ID NO: 27 - DNA, 40 bp, Artificial Sequence
  Synthetic oligonucleotide JS050
tcagtgccttgagtaacagtgcccgtataaacagttaatg

SEQ ID NO: 28 - DNA, 40 bp, Artificial Sequence
  Synthetic oligonucleotide JS051
aagtcagagggtaattgagcgctaatatcagagagataac

SEQ ID NO: 29 - DNA, 43 bp, Artificial Sequence; identical to SEQ ID NO: 19
  Synthetic oligonucleotide JS065

SEQ ID NO: 30 - DNA, 24 bp, Artificial Sequence
  Synthetic oligonucleotide JS045
tccggccttgctaatggtaatggt

SEQ ID NO: 31 - DNA, 24 bp, Artificial Sequence
  Synthetic oligonucleotide JS046
acattcaaccgattgagggaggga

SEQ ID NO: 32 - DNA, 24 bp, Artificial Sequence
  Synthetic oligonucleotide JS047
aacgcgctacagtctgacgctaaa

SEQ ID NO: 33 - DNA, 43 bp, Artificial Sequence; identical to SEQ ID NO: 18
  Synthetic oligonucleotide JS044

SEQ ID NO: 34 - DNA, 24 bp, Artificial Sequence
  Synthetic oligonucleotide JS040
tgaagatcagttgggtgcacgagt

SEQ ID NO: 35 - DNA, 24 bp, Artificial Sequence
  Synthetic oligonucleotide JS041
aatagtgtatgcggcgaccgagtt

SEQ ID NO: 36 - DNA, 24 bp, Artificial Sequence
  Synthetic oligonucleotide JS042
aactcggtcgccgcatacactatt

SEQ ID NO: 37 - DNA, 24 bp, Artificial Sequence
  Synthetic oligonucleotide JS043
tgtcagaagtaagttggccgcagt

SEQ ID NO: 38 - DNA, 19 bp, Artificial Sequence
  Synthetic oligonucleotide JS119
ggcaaggccggatccagac

SEQ ID NO: 39 - DNA, 21 bp, Artificial Sequence
  Synthetic oligonucleotide JS126
gtcaggttcaccgatggagcg

SEQ ID NO: 40 - DNA, 98 bp, Artificial Sequence
  Synthetic oligonucleotide JS140
gtccgctcgagacaccgaaaacggtgtctcgagcggaccactgactgtatgatgaagatgctgacgaggtctggatccggccttgccgcagctggtac

SEQ ID NO: 41 - DNA, 50 bp, Artificial Sequence
  Synthetic oligonucleotide JS141
gcacgtcaggcacggcgtcggtaccagctgcggcaaggccggatccagac

SEQ ID NO: 42 - DNA, 30 bp, Artificial Sequence
  Synthetic oligonucleotide CPD oligomer
ctcgtcagcatcttcatcatacagtcagtg

(19) United States
(12) Patent Application Publication
(10) Pub. No.: US 2013/0115598 A1
(43) Pub. Date: May 9, 2013

(54) OLIGONUCLEOTIDE PROBE RETRIEVAL ASSAY FOR DNA TRANSACTIONS IN MAMMALIAN CELLS

(71) Applicants: Lawrence Loeb, Seattle, WA (US); Jiang-Cheng Shen, Seattle, WA (US); Edward J. Fox, Seattle, WA (US)

(72) Inventors: Lawrence Loeb, Seattle, WA (US); Jiang-Cheng Shen, Seattle, WA (US); Edward J. Fox, Seattle, WA (US)

(21) Appl. No.: 13/652,431

(22) Filed: Oct. 15, 2012

Related U.S. Application Data
(60) Provisional application No. 61/546,899, filed on Oct. 13, 2011.

(51) Int. Cl. C12Q 1/68

(52) U.S. Cl. CPC C12Q 1/6827
USPC 435/6.11

(57) ABSTRACT

Methods to measure a variety of DNA synthetic processes in live human cells by introducing and retrieving exogenous DNA probes are provided herein. Using fragments of bacterial plasmid or phage DNA, a wide array of DNA constructs may be assembled to mimic the intermediates of DNA transactions, including replication, translation synthesis, and end-joining. These DNA probes may be transfected into human cells and retrieved for mutational analysis using a modified Random Mutation Capture assay or NextGen DNA sequencing. These assays require only a small number of cells, such as might be available from biopsy material. Thus, the methods described herein may be applied to the early detection of cancer, predicting the responsiveness of individual cancers to chemotherapy, and measuring the DNA repair capacity of individuals to environmental DNA damaging agents. This approach may be automated and used for screening human populations for variations in DNA synthetic and repair activities.

### PRIORITY CLAIM

The present application claims priority to U.S. Provisional Application No. 61/546,899, filed on Oct. 13, 2011, the subject matter of which is hereby incorporated by reference as if fully set forth herein.

### STATEMENT OF GOVERNMENT INTEREST

The present invention was made with government support under Grant Nos. CA-102029, CA115802, CA077852 and AG00751 awarded by the National Institutes of Health. The Government has certain rights in the invention.

### BACKGROUND

The human genome is subject to constant attack by endogenous reactive molecules and environmental agents, with as many as 50,000 DNA base modifications introduced per cell per day (Lindahl & Wood 1999). In addition, replication errors can account for thousands of base changes per cell per day (McCulloch & Kunkel 2008). To maintain genomic integrity, cells have developed multiple DNA repair mechanisms with overlapping specificities that excise damaged nucleotides in DNA (Friedberg et al. 2005; Barnes & Lindahl 2004) and correct polymerase errors (Liu et al. 2010). Somatic mutations still accumulate, leading to gene dysfunction that can result in multiple human diseases including cancer (Loeb 2011).

The causal association of mutations in DNA repair genes with inherited human diseases was first documented in xeroderma pigmentosum (XP) (Cleaver 2005), and extended to diseases predisposing to malignancy (D'Andrea & Grompe 2003), and developmental abnormalities (Spivak 2004; Newman et al. 2006), and neurological dysfunctions (McKinnon 2009). In contrast, somatic mutation and altered function of DNA repair genes have been much more difficult to establish. To date, there have not been versatile assays with which one can interrogate cells from multiple individuals and stratify them with respect to different disease states. Furthermore, there is a need to be able to assess the activity of different DNA repair pathways in modulating the response of individuals to chemotherapeutic agents (Helleday et al. 2008).

Direct measurements of catalytic activities of specific DNA repair enzymes in cell lysates have been used to identify repair deficient phenotypes (Wood et al. 1988; Feng et al. 2004). These assays have been limited however, as only certain cell types can be studied and enzyme kinetics measured in vitro may not be indicative of in vivo activities. In vivo methods such as the Comet assay (Collins 2004; Wu et al. 2005), Unscheduled DNA Synthesis (UDS) (Cleaver & Thomas 1981; Latimer et al. 2010) and Host Cell Reactivation assay (HCR) (Hy et al. 2004; Wang et al. 2010; Spivak & Hanawalt 2006) have also been used but are labor intensive and costly. Both the Comet and UDS assays require exogenous treatment of host cells with DNA damaging agents, such as UV irradiation, to induce repair. HCR involves transfection of host cells with engineered bacterial plasmids that contain a site specific lesion or pretreatment with DNA damaging agents.

Accumulation of mutations is a general characteristic of human cancers (Hanahan & Weinberg 2011; Loeb 2011), with specific mutations underlying many individual cancer phenotypes. Several methods exist for measuring mutation frequencies of genomic DNA in mammalian cells. Most of these methods identify alterations in hemizygously encoded genes (i.e., located on the X-chromosome) and measure either reversions of mutations or mutations that render the sentinel gene nonfunctional. Among the most widely used approaches are studies with HPRT (Albertini et al. 1990) and PIG-A (Chen et al. 2001). These approaches are labor-intensive and many require the establishment of cell lines. They are also each limited to a specific gene and a specific DNA sequence. Plasmid surrogates, such as pSP189 that can replicate in mammalian cells have also been used to measure mutation frequency in replicating cells (Kim & Wogan 2006; Lee & Pfeifer 2008). The sensitivity of these assays is often limited by low transfection efficiency and insufficient recovery of the plasmid for subsequent bacterial transformation. The analysis of DNA repair and replication in the human population has thus been limited by the lack of simple and versatile assays and an improvement in this area would be a significant advancement.

### SUMMARY

In some embodiments, methods of measuring at least one DNA integrity transaction (e.g., DNA repair, DNA mutation, and/or DNA replication fidelity) in a mammalian cell are provided. Such methods may include transfecting the cell with a plurality of oligonucleotide probes, the probes being blocked at the 5′ end. the 3′ end, or both, and comprising a tagged residue; retrieving the plurality of oligonucleotide probes by targeting the tagged residue; and quantifying the DNA integrity transaction.

In other embodiments, methods of determining a personalized cancer treatment for a cancer patient are provided. Such methods may include measuring a repair capacity of a cancer cell derived from the cancer patient in response to treatment with an effective amount of one or more chemotherapeutic agents; determining a responsiveness of the cancer cell to the one or more chemotherapeutic agents; and selecting one or more chemotherapeutic agents that are responsive in the cancer cell for the personalized cancer treatment, wherein (a) the cancer cell is responsive to the one or more chemotherapeutic agents when the repair rate is low; and (b) the cancer cell is not responsive to the one or more chemotherapeutic agents (e.g., a nucleoside analog) when the repair rate is high. The repair rate may be measured by a method that includes transfecting the cell with a plurality of oligonucleotide probes, the probes being blocked at either or both the 5′ and 3′ ends and comprising a tagged residue; retrieving the plurality of oligonucleotide probes by targeting the tagged residue; and quantifying the DNA repair rate.

In other embodiments, methods of determining the ability of a putative mutagen to induce a DNA mutation in a mammalian cell are provided. Such methods may include administering an effective amount of the mutagen to the cell; measuring a DNA mutation frequency or DNA repair rate in the cell; and determining that (a) the mutagen is effective at inducing a mutation when the DNA repair rate is low or the DNA mutation rate is high; and (b) the mutagen is not effective at inducing a mutation when the DNA repair rate is high or the DNA mutation rate is low. In some aspects, the cell may part of a population of cells taken from a plurality of similarly situated subjects.

### BRIEF DESCRIPTION OF THE DRAWINGS

FIG. 1. Nucleotide Excision Repair activities in vivo. (a) Construction of the OPRA probe that contains a cyclobutane pyrimidine dimer (CPD) lesion. The 30-nucleotide (nt) synthetic ssDNA fragment containing a CPD lesion (marked as a T-T dimer) was hybridized with a 5′-biotinylated and a 3′-ddC protected fragments to form a hairpin dsDNA construct after ligation. Primers for qPCR quantification are shown in arrow lines, where amplification by primers a/b represents the total amount of the probe and by a/c the amount of the repaired probe. The percentages of probe repaired were calculated by the following equation: % Repaired=(2^{-ΔCt})×100, ΔCt=Ct_{Control(a/b)}−Ct_{Test(a/c)}. (b) NER capacities in a single 293T cell are presented as % CPD probe repaired at incremental time points. Data are presented as mean±S.D. from three independent experiments. (c) NER capacities of human SV40-transformed, normal (GM00637) versus XPA (GM04429) fibroblast cells. Data are presented as mean±S.D. (n=3) and the P value was calculated by Student's t-test. (d) NER capacities of human primary fibroblasts. Human primary fibroblast cells, normal (AG01440) versus XPA (AG06971), were transfected with CPD probes and incubated at 37° C. for 48 h and 96 h prior to oligonucleotide probe retrieval. Data derived from qPCR analyses of the retrieved probes from three independent experiments are presented as % repaired of the CPD probes per cell (mean±S.D., n=3). The P value was obtained by Student's t-test. The absolute values likely reflect differences in kinetics of repair between primary and immortalized cells.

FIG. 2. Schematic diagram of the Oligonucleotide Probe Retrieval Assay (OPRA). Terminally blocked DNA probes, including blunt-ended dsDNA, primer/template partial duplex, D-loop structure, lesion-encoded primer/template and restricted plasmid DNA fragment, were used to transfect human cells.

FIG. 3. The Oligonucleotide Probe Retrieval Assay (OPRA). (a) Transfection efficiencies (copies per cell) of synthetic oligonucleotide probes in HEK293T cells. HEK293T cells (1×10⁶) were transfected using calcium phosphate complexes with different concentrations of the oligonucleotide probe (0, 2, 5 and 10 nM) and incubated at 37° C. for 16 h, followed by probe retrieval and qPCR calculation (see Materials and Methods). The controls are transfections in the absence of CaCl₂. Data are presented as mean±S.D. from two independent experiments. Numbers shown in parentheses are the mean of the probe molecules retrieved from each cell. (b) Stability of DNA probes in HEK293T cells. HEK293T cells (1×10⁶) were transfected with 5 nM of oligonucleotide probe and incubated at 37° C. The dsDNA probe transfected cells were harvested for DNA extraction at 16 h, 24 h, 48 h and 72 h after transfection. The mock control was cells transfected in the absence of CaCl₂ and harvested at 16 h. The copy number of the retrieved probe per cell was quantified by qPCR. The results are presented as mean±S.D. from two independent experiments. Stability of the probe is presented as percentage of probe retained at different time points relative to the 16 h incubation.

FIG. 4. Construction of the primer extension probe using M13 ssDNA as template. M13mp18 ssDNA was first hybridized with primers at restriction sites AfeI and BaeGI and then digested with designated restriction enzymes to produce a single-stranded linear DNA fragment. The linear DNA was used as a template to hybridize with a 5′-biotinylated oligonucleotide containing the unique barcode sequence for primer elongation.

FIG. 5. A flowchart for in vivo primer extension, DNA probe retrieval and mutational analysis. The 5′-biotinylated probes retrieved from the transfected cells were digested with TaqI (TCGA) and then enriched by magnetic beads conjugated to streptavidin. After removing the complementary strands by 5′-exonuclease degradation and heat denaturation, the adhered 5′-biotinylated strands were released from the beads for qPCR analysis using a primer set with one primer containing the unique barcode sequence.

FIG. 6. Primer extension in vivo. (a) Mutation frequencies of the elongated M13 primer/template in HEK 293T cells. In addition to the primer extension probe, the cells were also transfected with a linear M13 DNA fragment alone, as a reference for spontaneous DNA damage. (b) Mutation frequencies of primer extension in human primary fibroblasts (Normal Human Dermal Fibroblasts, NHDF). Data are presented as mean±S.D. from two independent experiments and statistics were obtained by t-test.

FIG. 7. Assessment of mutations induced by nucleoside analogs in human cells using OPRA. (a) A schematic diagram illustrating the assay. Cells were incubated with nucleoside analogs before transfection by Lipofectamine 2000 with the M13 primer/template probe. After incubation, the probes were retrieved from the transfected cells and the 5′-biotinylated strands containing the incorporated nucleoside analogs were isolated by streptavidin biotin capture. This DNA strand was then hybridized to a primer at the 3′-end and copied by Taq DNA polymerase. The copied strand was separated from the biotinylated strand by heat denaturation and then PCR amplified. The amplicons were digested with TaqI and the products subjected to the modified RMC analysis. (b) Increased mutation frequencies in DNA probes retrieved from 293T cells treated with nucleoside analogs 2-aminopurine (2-AP) and 2′-deoxyinosine (dl). Data are presented as mean±S.D. from three independent experiments and statistics were obtained by t-test.

FIG. 8. Primer extension in a D-loop structure. (a) Construction of D-loop DNA. Plasmid pCEP4 was digested with AatII and the resulting products were hybridized with a designated 5′-biotinylated primer that allows DNA copying through a TCGA target site. The formed triplex DNA mimicking a D-loop construct was transfected into HEK293T cells and the probe was retrieved after incubation for qPCR analysis by the modified RMC protocol. (b) Mutation frequencies of the retrieved D-loop probes from transfected HEK 293T cells. In addition to the D-loop probe, the DNA fragment without hybridization with the primer was also transfected to HEK293T cells and served as a reference for spontaneous mutation of the template. Data are presented as mean±S.D. from two independent experiments and statistics were obtained by t-test.

FIG. 9. OPRA for measuring Nucleotide Excision Repair. (a) Calibration of CPD probe. qPCR reaction was carried out by using 1 μl of 5 nM ligated CPD probe or normal TT probe as template. PCR amplifications by primers a and b are the internal control for the total number of the substrates, whereas amplifications by primers a and c represent the number of the repair products. Based on the ΔCt value (ac−ab) of the TT probe, the ligation efficiency (% ligation=1/2^{ΔCt}×100) was 1.5%. Since CPD adduct blocks the activity of Taq polymerase, the ΔCt value (ac−ab) of the CPD probe is greater than that of the normal TT control, yielding a ΔΔCt value (CPD-TT) of 10.71 that converts to a 1.670-fold blockage by CPD (Fold Difference=2^{ΔΔCt}). Using this 2^{ΔΔCt} method, differential repair efficiencies of CPD probes were obtained in cells with altered genetic backgrounds. Data are presented as mean±S.D. from three independent experiments. (b) Repair of CPD oligonucleotides in Human Embryonic Kidney Cell 293T. HEK293T cells (5×10⁵) were transfected with 0.02 nM of CPD probe and incubated at 37° C. Cells were harvested at increasing incubation times, followed by probe retrieval and qPCR analysis. Data are shown as the number of probes retrieved per cell and the number of probes repaired per cell at different time points from three independent experiments (mean±S.D.).

FIG. 10. Analysis of DNA end-joining events in cells by OPRA. (A) Protocol for assaying DNA end-joining in cells using a plasmid DNA fragment. Plasmid pCEP4 was digested with SalI and the resulting DNA fragments were gel purified. The purified 8.9 kb fragment with the Sail-cleaved, 5′-protruding TCGA sequence at both termini, was used to transfect HEK293T cells. After incubation and rejoining of the termini, the rejoined DNA molecules were extracted from the cells and quantified by real-time quantitative PCR (qPCR) at single molecule resolution. The qPCR amplicons of the single molecule were sequenced to reveal the context of the conjoined region. (B) DNA sequences of the conjoined region of the joined molecules retrieved from HEK 293T cells (SEQ ID NOs 1-12). The arrow indicates the cleavage site by restriction enzyme SalI, and the sequence (TCGA) of the coherent end is underlined. The original sequence of the SalI site (GTCGA) is marked by (*). The inserted bases at the conjoined region are circled and the deleted bases are presented either in lower case in a parenthesis or by a dash. At least four possible mechanisms can be deduced from the observed end-joining products. 1. Direct cohesive end ligation (3 of 12; A3, C2 and C8), which results in restoration of the wild-type SalI sequence GTCGAC. 2. Filled-in blunt-end ligation (3 of 12; A10, A12 and C10), which involves DNA synthesis on both termini and results in a repeat of TCGA in the SalI site. 3. Partially filled-in end ligation (2 of 12; D10 and D12), which may involve both Flap-endonuclease activities and partial filled-in DNA synthesis on one terminus, resulting in insertion of either TC or GA of the TCGA sequence in the SalI site. 4. Recessed blunt end ligation (4 of 12; B2, B4, B11 and C5), which results in deletion of the SalI site.

FIG. 11. Assaying translesion DNA synthesis in vivo using oligonucleotide probes. (A) The efficiencies of bypassing 8-oxo-dG in HEK 293T cells. The bypassing efficiencies are presented as the percentage primers elongated past the target G=O or G residue of the total probes retrieved from the transfected cells. The bypass percentage of each DNA probe is shown in parentheses. Data are presented as mean±S.D. from two independent experiments. (B) The error frequencies of bypassing 8-oxo-dG in HEK 293T cells. The percentage of bypassing error for each probe is shown in parentheses. Data are presented as mean±S.D. from two independent experiments and statistics were obtained by t-test. (C) The efficiency of bypassing CPD was 20-fold less efficient than bypassing canonical TT.

### DETAILED DESCRIPTION

Methods of measuring DNA integrity transactions in a mammalian cell are provided herein. In some aspects, the mammalian cell is a human cell. DNA integrity transactions involve one or more steps or processes that are responsible for maintaining the integrity of DNA in a cell. A DNA integrity transaction may refer to, but is not limited to, DNA repair events (e.g., repair rate, repair capacity, or repair accuracy), DNA mutation events (e.g., recombination, mutation rate or mutation frequency), DNA mutagenicity (i.e., likelihood of mutation when exposed to a mutagen), and DNA replication fidelity (i.e., likelihood of mutation during DNA synthesis). Thus, the methods described herein, which may be referred to as an Oligonucleotide Probe Retrieval Assay (OPRA), may be used to quantify spontaneous or induced mutations (e.g., tautomerism, depurination, deamination, single nucleotide polymorphisms (SNPs), recombination, or incorporation of nucleobase analogues) or mutagenicity, DNA repair and the fidelity of DNA synthetic processes in live human cells. In addition, OPRA may be used in applications such as early detection of cancer, predicting the responsiveness of individual cancers to chemotherapy, and measuring the DNA repair capacity of individuals to environmental DNA damaging agents. In addition, these methods may be automated and used for screening human populations for variations in DNA synthetic and repair activities.

In some embodiments, the methods described herein include a step of transfecting the cell with a plurality of oligonucleotide probes. In some embodiments, the oligonucleotide probes are synthetic probes, and may be blunt-ended dsDNA probes, primer/template partial duplex probes, D-loop structure probes, lesion-encoded primer/template probes, restricted plasmid DNA fragment probes, or a combination thereof. In other embodiments, the oligonucleotide probes are derived from natural DNA (i.e., isolated from one or more living organisms including, but not limited to, phages, bacteria, and mammalian cells).

A large number of molecules or probes can be introduced and retrieved per cell. In certain embodiments, the plurality of oligonucleotide probes may include as few as approximately four oligonucleotide probes, and as many as approximately 200,000 oligonucleotide probes. In some aspects, the plurality of oligonucleotide probes may include approximately 10,000 oligonucleotide probes, approximately 20,000 oligonucleotide probes, approximately 30,000 oligonucleotide probes, approximately 40,000 oligonucleotide probes, approximately 50,000 oligonucleotide probes, approximately 60,000 oligonucleotide probes, approximately 70,000 oligonucleotide probes, approximately 80,000 oligonucleotide probes, approximately 90,000 oligonucleotide probes, approximately 100,000 oligonucleotide probes, approximately 200,000 oligonucleotide probes, less than 10,000 oligonucleotide probes, approximately 4 to 10,000 oligonucleotide probes, approximately 10,000 to 20,000 oligonucleotide probes, approximately 20,000 to 30,000 oligonucleotide probes, approximately 30,000 to 40,000 oligonucleotide probes, approximately 40,000 to 50,000 oligonucleotide probes, approximately 50,000 to 60,000 oligonucleotide probes, approximately 60,000 to 70,000 oligonucleotide probes, approximately 70,000 to 80,000 oligonucleotide probes, approximately 80,000 to 90,000 oligonucleotide probes, approximately 90,000 to 100,000 oligonucleotide probes, approximately 100,000 to 2000,000 oligonucleotide probes, more than 100,000 oligonucleotide probes, or more than 200,000 oligonucleotide probes.

For evaluating repair capacity, OPRA employs a linear DNA construct containing a site-specific DNA lesion that blocks qPCR; the lesion can, however, be repaired in a repair-proficient cell, resulting in an amplifiable product. For assaying replication fidelity, OPRA utilizes biologically derived DNA sequences to determine the frequency of mutations at single molecule resolution. The assay utilizes primers hybridized to defined DNA template sequences, which following transfection into human cells, can be recovered and assayed for mutations using a modification of the Random Mutation Capture (RMC) assay (Bielas and Loeb, 2005), permitting a level of mutation detection as low as 10⁻⁷ per base pair. As described in the example below, OPRA's versatility is demonstrated by constructing a series of DNA probes for assaying replication, recombinational DNA synthesis, translation DNA synthesis, DNA end-joining and nucleotide excision repair (NER). This is not the limit of the assay, as probes can be developed to investigate other DNA integrity transactions, as described further below.

The oligonucleotide probes may be blocked (i.e., non-cleavable) on the 3′-termini, 5′-termini, or on both 3′- and 5′-termini. In some embodiments, the probes may include a tagged residue (e.g., a barcode or other unique sequence) to assist with retrieval (described below). In some embodiments, the tagged residue is specific to a particular DNA integrity transaction to be measured. In other words, more than one DNA integrity transaction may be measured by using a first unique tagged residue for measuring DNA repair events, a second unique tagged residue for measuring DNA repair events, a third unique tagged residue for measuring replication fidelity, and so on. In this manner, a single assay or kit may be employed to measure multiple DNA integrity transactions.

In some embodiments, the methods described herein include a step of retrieving the oligonucleotides described above. In some aspects, the retrieval of the oligonucleotides may be accomplished by targeting the tagged residue using an avidin-biotin interaction, affinity chromatography, or any other suitable method known in the art. For example, in one embodiment, each of the oligonucleotide probes are introduced into a variety of cells including difficult-to-transfect cells, such as primary human fibroblasts and explant cultures, by transient transfection. Each transfected probe may contain one or more biotin residues and is retrieved by conjugation with streptavidin beads. The mutation frequencies in the de novo synthesized regions of the retrieved probes are determined by a modified RMC assay, DNA sequencing, or any other suitable method known in the art. Tens of thousands of oligonucleotides probes can be introduced into a single cell and retrieved efficiently for mutational analysis. An assortment of DNA constructs can be used in this system to query the error rates of the designated DNA transactions in human cells.

In some embodiments, the methods described herein include a step of quantifying the DNA integrity transaction, wherein the DNA integrity transaction involves one or more steps or processes that are responsible for restoring the integrity of DNA in a cell. In one embodiment, the DNA integrity transaction is quantified using any suitable polymerase chain reaction (PCR) method (e.g., real time quantitative PCR). In another embodiment, the DNA integrity transaction is quantified using a Random Mutation Capture (RMC) assay or a modified RMC assay, which are described in detail below.

According to some embodiments, the methods described herein may be used to determine a cancer patient's responsiveness to a chemotherapeutic agent. Such methods may be used to determine a personalized cancer treatment or treatment regimen for the cancer patient based on the responsiveness. In some embodiments, these methods may include a step of measuring a repair rate or repair capacity of a cancer cell derived from the cancer patient in response to treatment with an effective amount of one or more chemotherapeutic agents. In some aspects, the one or more chemotherapeutic agents are incorporated into a plurality of oligonucleotide probes used in the method to measure the repair capacity, as discussed below. In some embodiments, the repair capacity is measured by a method comprising transfecting the cell with a plurality of oligonucleotide probes, the probes being blocked at the 5′ end, the 3′ end, or both, and comprising a tagged residue; retrieving the plurality of oligonucleotide probes by targeting the tagged residue; and quantifying the DNA repair capacity.

In some embodiments, the methods for determining a cancer patient's responsiveness to one or more chemotherapeutic agents or methods for determining a personalized cancer treatment include as step of determining a responsiveness of the cancer cell to the one or more chemotherapeutic agents. Further, according to some embodiments, once the responsiveness to the one or more chemotherapeutic agents has been determined, the method may include selecting one or more chemotherapeutic agents that are responsive in the cancer cell for the personalized cancer treatment.

Many chemotherapeutic agents kill cancer cells by causing lethal DNA mutations or cause DNA damage that may lead to a lethal mutation. As such, the efficacy of these agents are dependent on the cell's ability to repair such mutations: if a cell exhibits a high repair rate as compared to a control cell or a diagnostic standard, or exhibits a high rate that prevents a mutation from being lethal, the chemotherapeutic agent is not likely to be effective and an alternative treatment should be pursued. Similarly, if a cell exhibits a low or normal repair rate as compared to a control cell or a diagnostic standard, or exhibits a rate that allows a lethal mutation to occur, the chemotherapeutic agent is likely to be effective.

In one embodiment, the one or more chemotherapeutic agents analyzed are selected from any agent that may be incorporated into DNA. Examples of chemotherapeutic agents that may be analyzed or used in accordance with the methods described herein include, but are not limited to, alkylating agents (e.g., nitrogen mustards, nitrosoureas, alkyl sulfonates, triazines, and ethylenimines); antimetabolites (e.g., 5-fluorouracil (5-FU), capecitabine, 6-mercaptopurine (6-MP), methotrexate, gemcitabine, cytarabine, fludarabine and pemetrexed); platinum drugs (cisplatin, carboplatin, and oxalaplatin); nucleoside analogs, nucleotide analogs, purine analogs, pyrimidine analogs or any other suitable nucleobase analog (e.g., didanosine, vidarabine, cytarabine, emticitabine, lamivudine, zalcitabine, abacavir, entecavir, stavudine, telbivudine, zidovudine, idoxuridine, trifluridine, 2-aminopurine (2-AP) or 2′-deoxyinosine (dl)); crosslinking agents; or any other DNA damaging agents.

According to some embodiments, quantification of the DNA repair capacity is accomplished by measuring the incorporation of the one or more chemotherapeutic agents into the oligonucleotide probe. Incorporation of the one or more chemotherapeutic agents into the probe can be measured by any suitable method known in the art including, but not limited to, mass spectrometry or a hybridization assay. In other embodiments, the one or more chemotherapeutic agents may be covalently linked to the oligonucleotide probes. In this case, quantification of the DNA repair capacity may be accomplished by the cancer cell's ability to excise (e.g., by NER) the one or more chemotherapeutic agent from the probe(s).

An “effective amount” or “effective dose” is an amount of a therapeutic agent (e.g., a chemotherapeutic agent) that produces a desired therapeutic effect in a subject or a cell, such as preventing or treating a target condition or alleviating symptoms associated with the condition. The precise therapeutically effective amount is an amount of the composition that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the cell or subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine an effective amount through routine experimentation, namely by monitoring a subject's or a cell's response to administration of a compound and adjusting the dosage accordingly. For additional guidance, see Remington: The Science and Practice of Pharmacy 21^{st} Edition, Univ. of Sciences in Philadelphia (USIP), Lippincott Williams & Wilkins, Philadelphia, Pa., 2005

The mammalian cells, the human cells, or the cancer cells used in the methods described herein may be derived from any biological sample containing DNA including, but not limited to, any material, biological fluid, tissue (including tumor tissue or normal tissue), circulating cell or cell obtained or otherwise derived from the cancer patient including, but not limited to, blood (including whole blood, leukocytes, peripheral blood mononuclear cells, buffy coat, plasma, and serum), sputum, tears, mucus, nasal washes, nasal aspirate, breath, urine, semen, saliva, meningeal fluid, amniotic fluid, glandular fluid, lymph fluid, milk, bronchial aspirate, synovial fluid, joint aspirate, cells, a cellular extract, and cerebrospinal fluid. This also includes experimentally separated fractions of all of the preceding. For example, a blood sample can be fractionated into serum or into fractions containing particular types of blood cells, such as red blood cells or white blood cells (leukocytes). If desired, a sample may be a combination of samples from an individual, such as a combination of a tissue and fluid sample. A biological sample may also include materials containing homogenized solid material, such as from a stool sample, a normal tissue or tumor tissue sample, or a tissue biopsy, micro-biopsy, tumor explant, circulating tumor cells; or materials derived from a tissue culture or a cell culture.

In some embodiments, the methods for determining a cancer patient's responsiveness to one or more chemotherapeutic agents or methods for determining a personalized cancer treatment include treating a cancer cell derived from the cancer patient with an effective amount of the one or more chemotherapeutic agents. The one or more chemotherapeutic agents may be administered on their own or in combination to the cancer cell.

In addition to the methods described above, the simplicity of this assay should facilitate epidemiological studies to establish the association of deficits in DNA repairs with disease incidence. Therefore, in another embodiment, the methods described herein may be used to determine the ability of a putative mutagen to induce a DNA mutation in a mammalian cell (e.g., a human cell). Such methods may include steps of administering an effective amount of the mutagen to the cell, measuring a DNA mutation frequency or DNA repair rate in the cell by the methods described herein. If the mutagen induces a mutation rate in the cell that is larger than a repair rate, the mutagen is likely effective in causing a mutation that may lead to cancer or another disease or condition caused by DNA mutation. Similarly, if the mutagen induces a mutation rate in the cell that is smaller or lower than a repair rate, the mutagen is not likely to be effective in causing a mutation that may lead to cancer or another disease or condition caused by DNA mutation. In some aspects, the cell may be part of a population of cells taken from a plurality of similarly situated subjects. In this manner, the methods may be used to stratify subjects by geographical area (i.e., subjects living in an area exposed to a particular environmental toxin or other agent), by diagnostic results (i.e., subjects having a particular gene defect) or by any other clinically relevant category.

In other embodiments, the methods provided herein may be used to establish or provide an index or reference standard for classification of different tumor types. In such embodiments, the methods may include measuring the fidelity of DNA synthesis in a plurality of cancer cells, which are derived from a plurality of tumor types. In other words, a subset of cells derived from a particular type of tumor may be analyzed by the methods provided herein, and said subset may be compared to a plurality of additional subsets analyzed in a similar manner. By analyzing cells from a plurality of tumor types, an index may be established that provides a reference error rate or range of error rates for each type of tumor, to which a cancer patient's cells may be compared in order to determine the progressiveness of the patient's tumor and the most effective treatment to administer the patient. According to these embodiments, the fidelity of DNA synthesis may be measured by a method comprising transfecting cancer cells from each tumor type with a plurality of oligonucleotide probes derived from natural DNA, wherein the probes include an unblocked 3′ end that is elongated in the cell and comprise a tagged residue; retrieving the plurality of oligonucleotide probes by targeting the tagged residue; and quantifying the DNA repair capacity by any suitable method described herein.

The methods described herein provide an improvement of previous methods for measuring DNA integrity transactions. As described in the studies below, OPRA provides a simple and cost-effective in vivo procedure for assaying the dynamics of mutation. By performing primer extension reaction in living cells and analyzing the contents of the elongated DNA from a 5′-tagged sequence, it is possible to directly capture the cellular products and intermediates of DNA synthesis. This assay measures the cell's potential to misincorporate during DNA polymerization, as opposed to merely cataloging the genetic aberrations that have accumulated over many generations and that are detected, for example, by direct sequencing of cellular DNA. In this regard, OPRA is particularly suited to characterize mutators during neoplastic development.

Two unexpected observations in the studies described below establish the versatility of the assay and the feasibility of measuring the fidelity of DNA synthetic processes in vivo. Firstly, due to the fact that a large number of molecules or probes (up to approximately 200,000 or more) can be introduced and retrieved per cell, it is feasible to use small samples of blood or biopsies for measuring DNA repair capacities. Secondly, while the high inaccuracy inherent in the chemical synthesis of oligonucleotides limits their utility for measuring the fidelity of DNA synthesis, it is shown that biologically derived DNA molecules, which are highly accurate, can be utilized to monitor the fidelity of a variety of different DNA transactions in cells.

Historically, the replication fidelity of individual DNA polymerases has been determined by in vitro biochemical assays using either enzymes purified directly from tissues or cell cultures, or recombinant proteins purified following ectopic expression in bacteria and yeast (Schmitt et al. 2010). As an indicator for replication fidelity, these biochemical assays have become gold standards by establishing kinetic constants for DNA polymerases copying designated DNA substrates in vitro (Arana & Kunkel 2010). To complement these single enzyme substrate reactions, cell extracts have also been used to assay replication fidelity.

Studying the biochemistry of DNA replication in this way has been important in estimating the potential of individual mutant polymerases to contribute to carcinogenesis. For example, mutant DNA polymerases lacking proof-reading capabilities or possessing compromised catalytic activities have been characterized biochemically (Patel et al. 2001; Schmitt et al. 2010); and subsequently analogous substitutions in DNA polymerases were demonstrated in animal models to be cancer-causing mutators (Albertson et al. 2009). Despite the power of biochemical assays for DNA polymerases, there are many important questions that cannot be approached by extrapolation from the results with purified molecules. In cells, DNA synthetic processes are carried out in complex environments at a total protein concentration approaching 300 mg/ml (Silverman & Glick, 1969). As a result DNA polymerases must be complexed with multiple proteins and mechanisms are required to bring these complexes to the sites of DNA synthesis. Furthermore, when replication is slowed or blocked there needs to be “hand-off mechanisms” to allow for synthesis by bypass DNA polymerases. OPRA allows the cellular transactions to be sampled in vivo with a mutational baseline of 3×10⁻⁷ and thus begins to approach the realities of chromosomal replication and repair. As proof of its utility, this approach was recently used to study another system: the effect of WRN depletion on the fidelity of de novo DNA synthesis in SV40-immortalized human fibroblast cells (Kamath-Loeb et al. 2012).

Previously, plasmid vectors harboring a reporter gene containing UV-induced photoproducts or oxidative damages have been successfully used in host cell reactivation assay for measuring the repair capacity in vivo (Ganesan et al. 1999; Spivak & Hanawalt 2006). Recently, a similar plasmid/reporter gene assay developed by Livneh et al. (Avkin & Livneh 2002; Ziv et al. 2009) has also successfully measured translational DNA synthesis in living cells and has been used to stratify individuals according to their susceptibility to mutagenesis by benzopyrene adducts (Avkin et al. 2004). However, the recovered plasmids need to be analyzed for mutations in indicator E. coli. Due to low transfection efficiency of plasmid DNA in mammalian cells, only hundreds to thousands of the subsequent E. coli transformants could be counted in each experiment. With a detection sensitivity of approximately 2-5×10⁻⁴, this assay thus may not be suitable for measuring the fidelity of DNA synthesis in human cells. In contrast, OPRA employs highly base-accurate plasmid or phage DNA fragments as a template for the primer run-off DNA synthesis and, with a mutational baseline of 3×10⁻⁷, (see Table 3 below) can recover as many as 1×10⁸ copies of the elongated DNA extracted from 1 million cells.

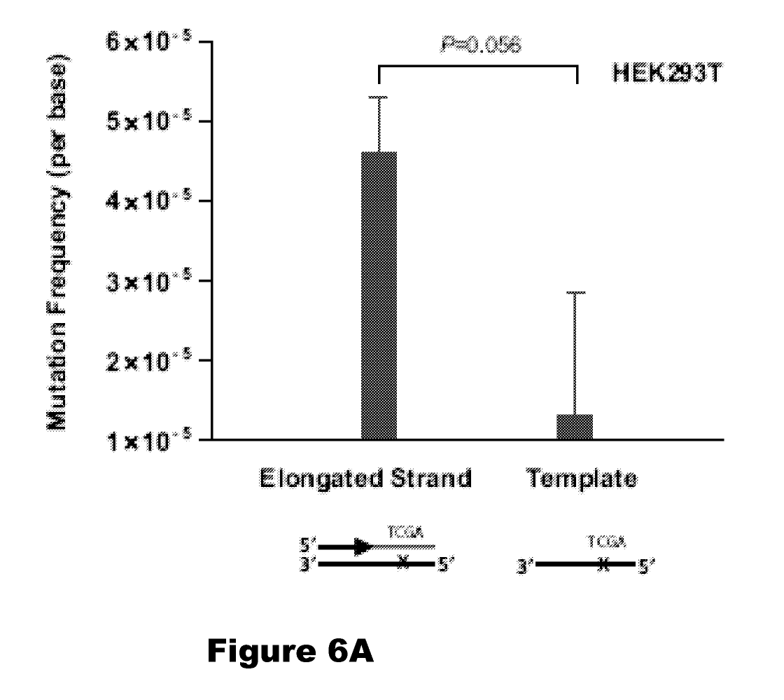

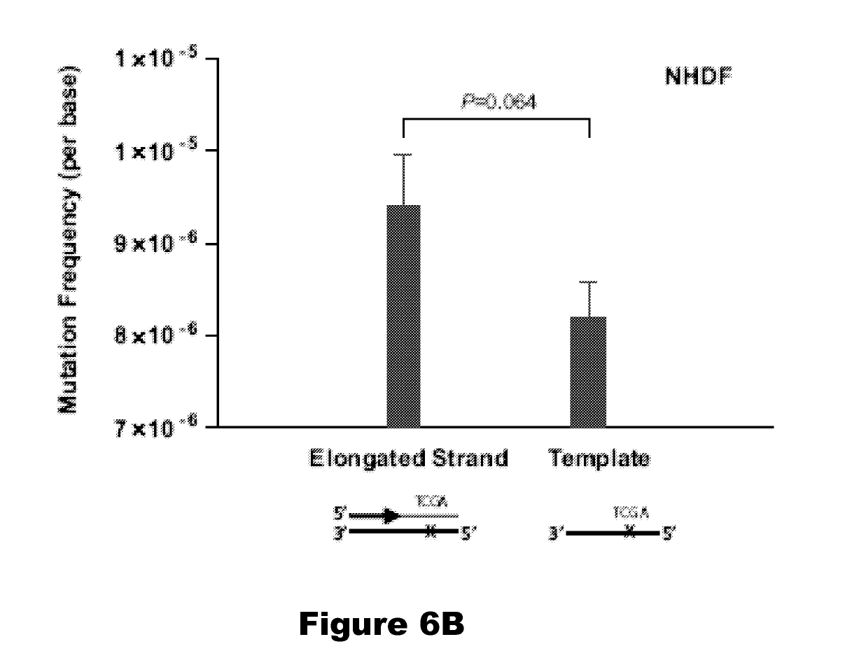

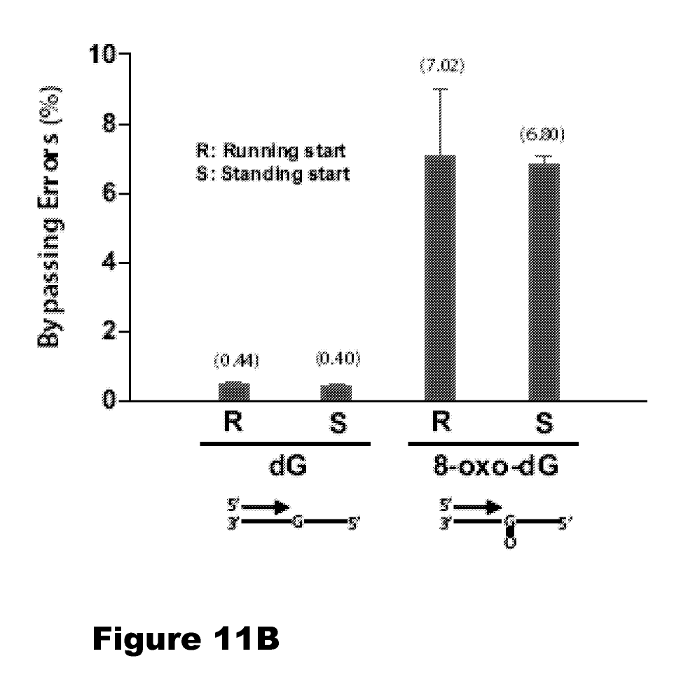

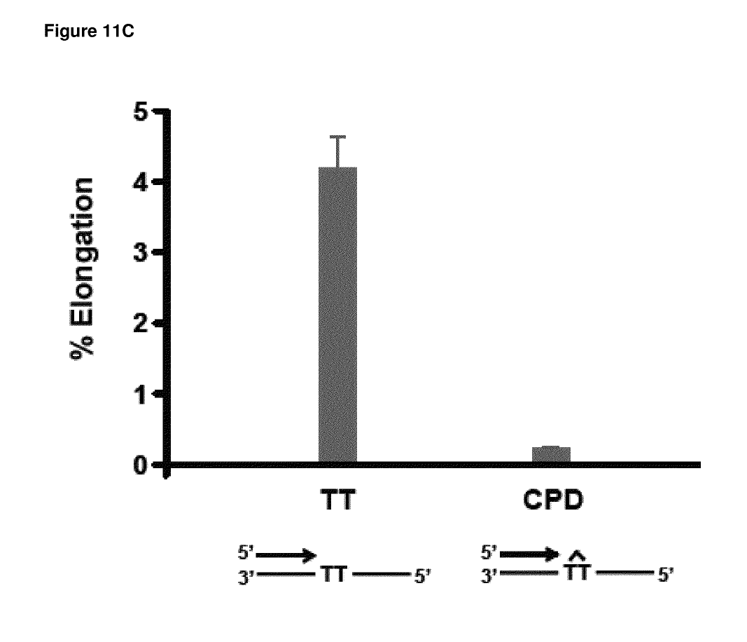

Although synthetic oligonucleotides are not feasible for measuring the fidelity of DNA synthesis owing to their high base-inaccuracy (1×10⁻³ per base as measured by the modified RMC assay, Table 3 below), they are suitable for assaying DNA repair, the efficiency of lesion bypass, and for measuring the error rate of base-pairing opposite specific lesion in human cells. The advantage of using synthetic primer/template oligonucleotides for assaying lesion bypass in cells is that cells can be transfected at a high concentration of DNA, at least 10-fold greater than the highest concentration achievable with the plasmid/phage based DNA probes. Due to high transfection efficiency with short oligonucleotides, about 200,000 copies of the DNA probe can be introduced into a single cell. 100 copies of the probe elongated past the lesion (see Table 1 below) and containing the predicted base substitution were retrieved. In the lesion-bypass probe, the 8-oxo-dG lesion is situated within the TaqI target site (TCGA) and thus can be analyzed for bypass-induced mutations using the modified RMC assay once the probe is retrieved. It was first demonstrated that the replacement of dG with 8-oxo-dG in the TaqI site does not block cleavage, and that TaqI efficiently cleaves ssDNA. Using an M13mp18 ssDNA fragment containing a TaqI site, TaqI digestion and subsequent qPCR was then used to determine that the cleavage efficiency for ssDNA derived from M13 phage is 1×10⁻⁶, while that of the M13-RF1 dsDNA is 3×10⁻⁷. Using the same digestion protocol, the error frequency of the synthetic G=O oligonucleotides was found to be 4.4×10⁻³ (see Table 3 below). Based on these characteristics, the fidelity of bypass of the 8-oxo-dG lesion in living cells was measured by the modified RMC assay. The results in 293T cells show that about 7% of the elongated primers mis-insert across from the 8-oxo-dG lesion (FIG. 11B). In order to measure bypass of adducts that less frequently mispair, it will be necessary to use natural DNA templates (FIG. 6). It should be noted that MYH may modify the bypassed strand when A is incorporated against 8-oxo-dG.

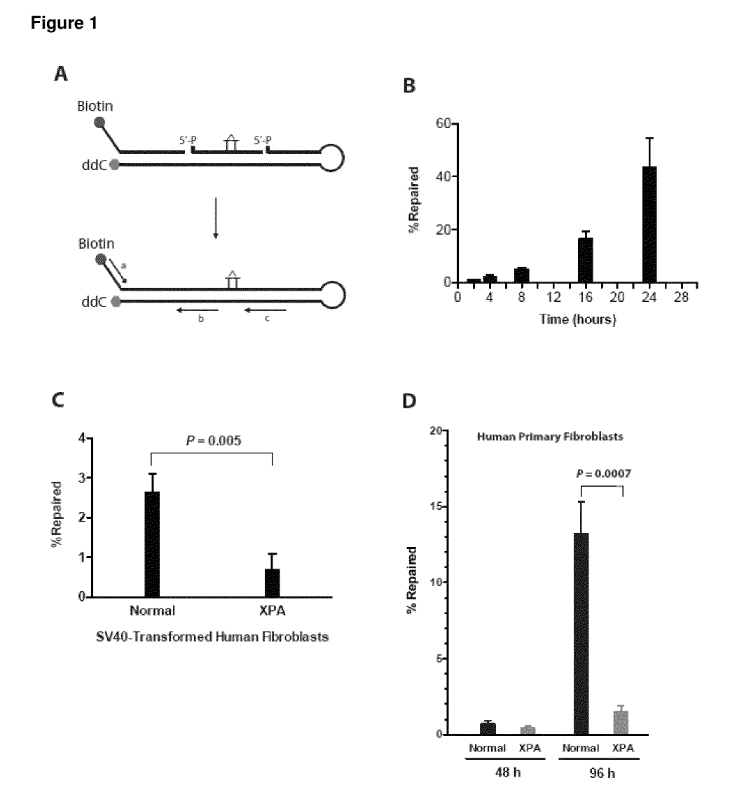

A major mechanism for the excision of large adducts from DNA is nucleotide excision repair (NER) (Cleaver et al. 2009). Customarily this repair process is monitored by either the comet assay (Collins 2004), unscheduled DNA synthesis (UDS) (Cleaver & Thomas 1981) or host cell reactivation (Spivak & Hanawalt 2006); these assays are labor intensive and difficult to quantitate. In contrast, OPRA is relatively easy to quantitate; it measures the end product of a completed NER process, unlike the comet assay which monitors only DNA breaks incised by NER nucleases or UDS which scores only the DNA synthesis during DNA repair. Because of the large amount of oligonucleotide probes that are introduced into a single cell, as well as the stability of the probe inside the cells, which is likely due to the end-protection and the hairpin structure, DNA repair can be monitored with a small amount of cells for as long as 96 hours (FIG. 1D). Furthermore, unlike the comet or UDS assay that requires microscopy and host cell reactivation that needs bacterial transformation, OPRA analysis of DNA repair can be quantified by qPCR (the 2^{-ΔΔCt} method). In addition, while it has been demonstrated that a 77 bp template is subject to repair by NER, it is possible that repair efficiency may be greater on a longer template. In addition to NER, other mechanisms of DNA repair may be analyzed in accordance with the methods described herein including, but not limited to, base excision repair (BER), mismatch repair (MMR), homologous recombination (HR), and non-homologous end joining (NHEJ).

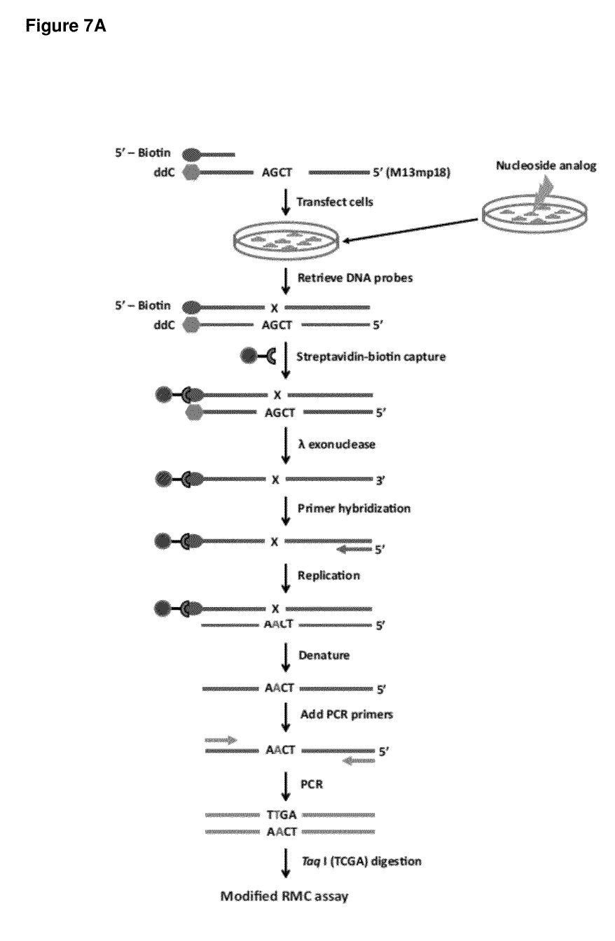

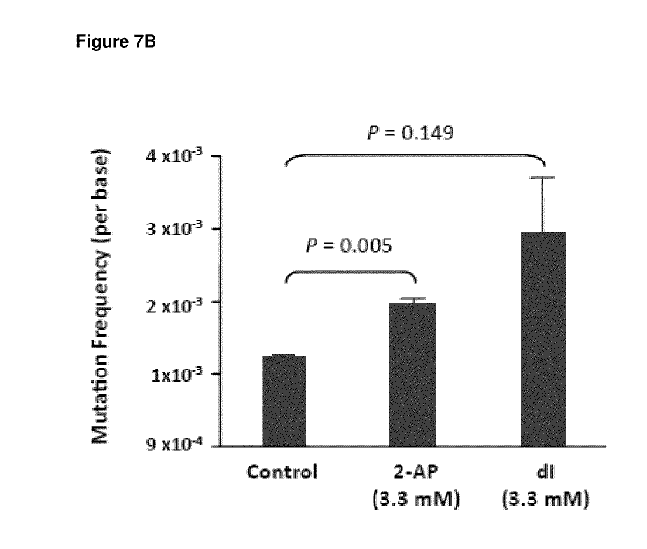

The in vivo primer extension assays can also be used to monitor the incorporation of mutagenic nucleoside analogs into DNA. Synthetic nucleoside analogs have proven to be effective therapeutic agents in treatment of cancers and in antiviral therapy. Nucleosides are metabolized in cells to nucleoside triphosphates and incorporated by cellular DNA polymerases. Due to their high frequency of mispairing with non-canonical bases during DNA replication, some of these nucleoside analogs are mutagenic. Therefore, assessing the mutagenicity of the analogs in living cells in terms of the metabolic rates of the compounds, the incorporation frequencies in DNA, the mispairing potentials in replication, and the end-point mutational profiles may be of use in their therapeutic development. It has been demonstrated here, using OPRA, that the elongated probe DNA retrieved from human embryonic kidney cells (293T) treated with nucleosides 2-aminopurine or deoxyinosine display increased mutations (FIG. 7B). Thus this assay can be used to rapidly and easily define the mutagenic footprint of nucleoside analogs in living cells.

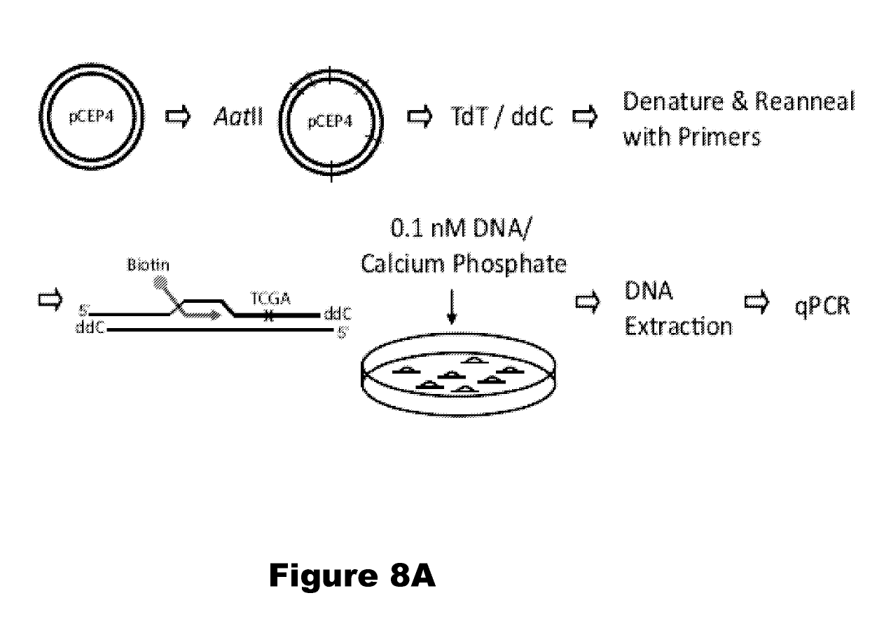

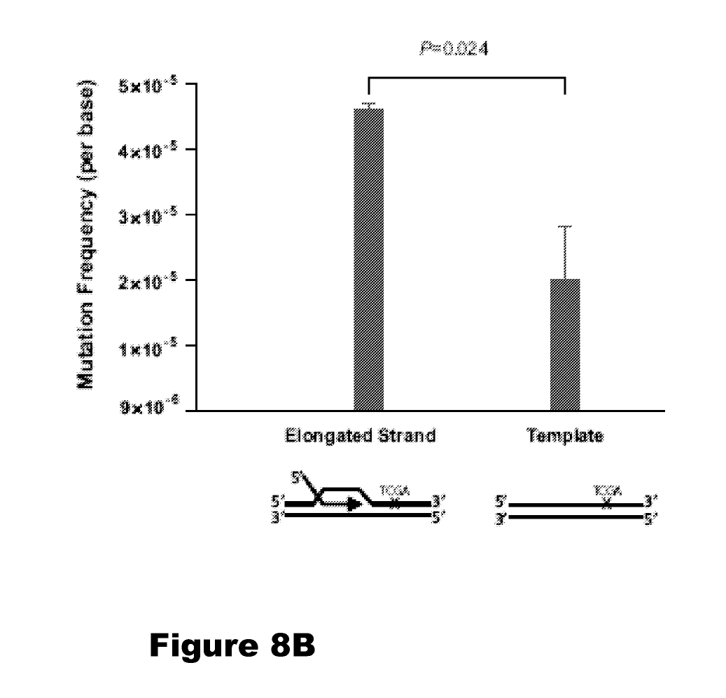

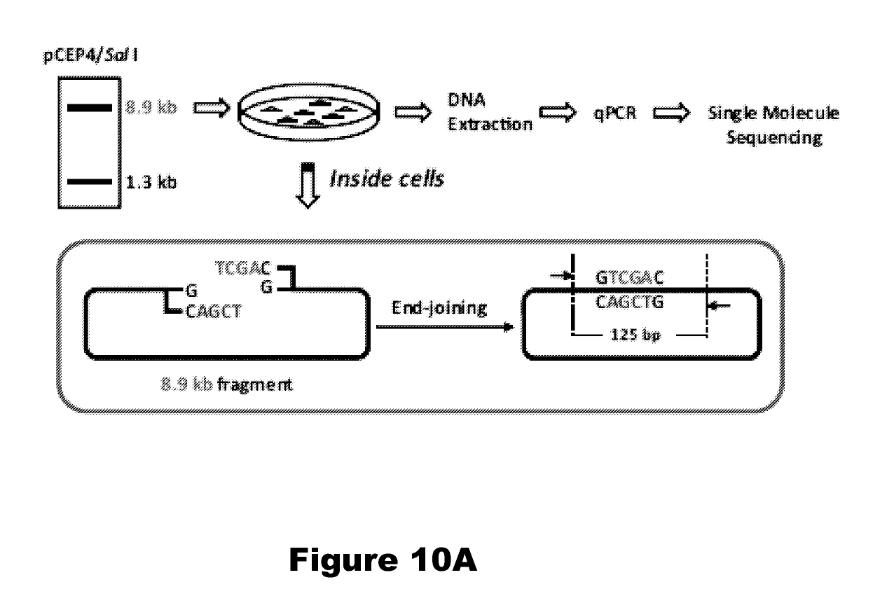

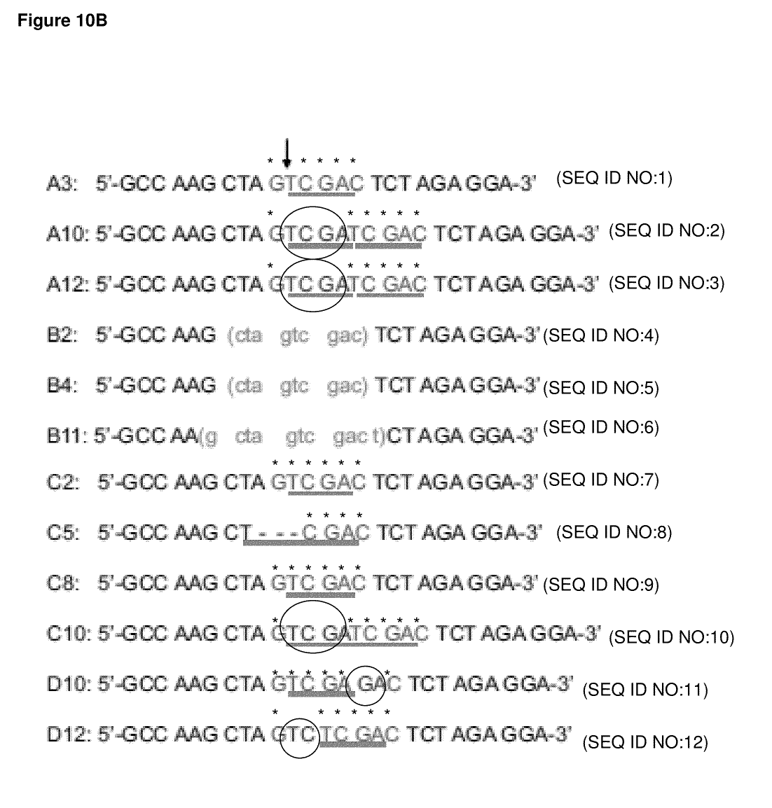

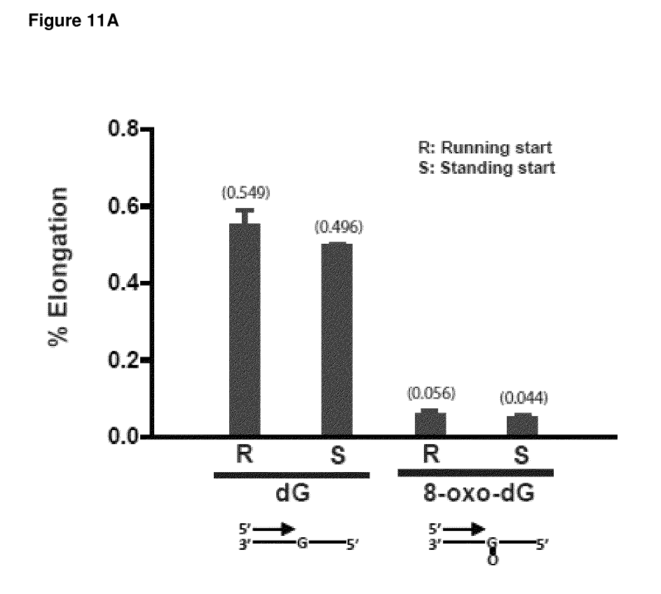

The copying of plasmid-derived DNAs and oligonucleotides in cells has invariably been shown to generate mutations at a higher rate than that occurs during the copying of chromosomal DNA (Bourre et al. 1989; Miller et al. 1984). Presumably mis-incorporations in these molecules are not subjected to mismatch correction. As a result, these measurements reflect the error rates of the relevant DNA polymerases; however this concept still needs to be vigorously established. Thus, similar to most other transient-based assay systems, the OPRA assays DNA synthesis that may not be subjected to mismatch correction. Furthermore, transient transfection approaches are generally unable to mimic the restrictions imposed by chromosomal structures in nuclei. Nevertheless they have provided important information about mutation frequency, spectra and participating DNA polymerases. In contrast to other approaches, tens of thousands of oligonucleotide probes can be introduced into a single cell and retrieved efficiently for mutational analyses. It is demonstrated that an assortment of DNA constructs, such as recombinational D-loop (FIG. 8) and DNA double-strand breaks (FIG. 10), can be used in this system to interrogate the fidelity of the designated DNA transactions in human cells.

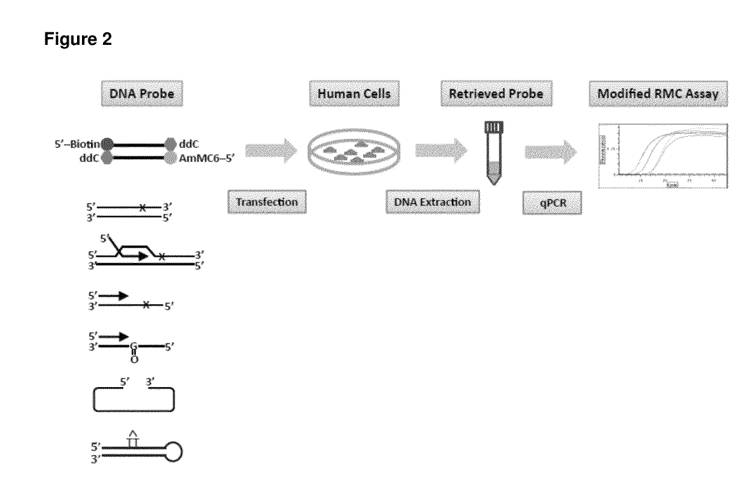

Different DNA constructs were used to demonstrate the versatility of OPRA in analyzing mutations caused by aberrant DNA transactions in human cells (FIG. 2). The conventional primer/template extension assay used to define the biochemistry of DNA polymerases can now be carried out inside cells. Based on the design of the DNA substrate, complex DNA transaction mechanisms can be analyzed and the involvement of different DNA polymerases in these processes can be established. Owing to the plasticity of this methodology; and its simplicity and feasibility for automation, it may be further utilized in studies of mutagenesis, genetic instability and tumor progression.

The following examples are intended to illustrate various embodiments of the invention. As such, the specific embodiments discussed are not to be construed as limitations on the scope of the invention. It will be apparent to one skilled in the art that various equivalents, changes, and modifications may be made without departing from the scope of invention, and it is understood that such equivalent embodiments are to be included herein. Further, all references cited in the disclosure are hereby incorporated by reference in their entirety, as if fully set forth herein.

### Example 1

### Methods for Measuring DNA Integrity Transactions

Materials and Methods

Oligonucleotides and Nucleoside Analogs.

Deoxyoligonucleotides were purchased from IDT or Midland (Table 1). Nucleoside analogs used in this report, 2-aminopurine and 2′-deoxyinosine, were purchased from Sigma. Both analogs were dissolved in DMSO to generate 100 mM stock solutions for applying to cell cultures. The 30-nt oligonucleotides containing site-specific CPD adduct was from Dr. S. Iwai (Osaka University, Osaka, Japan).

TABLE 1

| | | SEQ ID |
|---|---|---|
| Oligonucleotide | Sequence | NO. |
| JS001 | 5′-biotin-TAGCAAGCTTGCTAGCGGCCGCTCGAGGCCGGCAAGGCC | 13 |
| | GGATCCAGAC-ddC-3′ | |
| JS002 | 5′-AmMC6-GTCTGGATCCGGCCTTGCCGGCCTCGAGCGGCCGCTA | 14 |
| | GCAAGCTTGCTA-ddC-3′ | |
| JS011 | 5′-biotin-GCACGTCAGGCACGGCGTCCTGCTAGCAAGCTTGCTAGC | 15 |
| | GGCC-3′ | |
| JS025 | 5′-biotin-GCACGTCAGGCACGGCGTCTACGGGACTTTCCTACTTGG | 16 |
| | CAGT-3′ | |
| JS034 | 5′-biotin-GCACGTCAGGCACGGCGTCACTTACTCTAGCTTCCCGC | 17 |
| | AACA-3′ | |
| JS044 | 5′-biotin-GCACGTCAGGCACGGCGTCTGAAGATCAGTTGGGTGC | 18 |
| | ACGAGT-3′ | |
| JS065 | 5′-biotin-GCACGTCAGGCACGGCGTCACATTCAACCGATTGAGGG | 19 |
| | AGGGA-3′ | |
| JS122 | 5′-biotin-GCACGTCAGGCACGGCGTCAGCTGGGTACCAGCTGC | 20 |
| | TAGCAAGCTTGCTAGCGGCCGCTCGAGGCCGGCAAGGCCGGATC | |
| | CAGACATGATAAGATACATTGGCCACTGGACACCGTGTTG-3′ | |
| G = O template | 5′-biotin-GCACGTCAGGCACGGCGTCAGCTGGGTACCAGCTGCTAG | 21 |
| | CAAGCTTGCTAGCGGCCGCTCG=OAGGCCGGCAAGGCCGGATCC | |
| | AGACATGATAAGATACATTGGCCACTGGACACCGTGTTG-3′ | |
| JS124 | 5′-biotin-GTCAGGTTCACCGATGGAGCGCAATGTATCTTATCATGTC | 22 |
| | TGGATCCGGCCTTGCCGGCC-3′ | |
| JS125 | 5′-biotin-GTCAGGTTCACCGATGGAGCGCAATGTATCTTATCATGT | 23 |
| | CTGGATCCGGCCTTGCCGGCCT-3′ | |
| JS003 | 5′-TAGCAAGCTTGCTAGCGGCC-3′ | 24 |
| JS004 | 5′-GTCTGGATCCGGCCTTGCC-3′ | 25 |
| JS012 | 5′-GCACGTCAGGCACGGCGTC-3′ | 26 |
| JS050 | 5′-TCAGTGCCTTGAGTAACAGTGCCCGTATAAACAGTTAATG-3′ | 27 |
| JS051 | 5′-AAGTCAGAGGGTAATTGAGCGCTAATATCAGAGAGATAAC-3′ | 28 |
| JS065 | 5′-biotin-GCACGTCAGGCACGGCGTCACATTCAACCGATTGAGGGAG | 29 |
| | GGA-3′ | |
| JS045 | 5′-TCCGGCCTTGCTAATGGTAATGGT-3′ | 30 |
| JS046 | 5′-ACATTCAACCGATTGAGGGAGGGA-3′ | 31 |
| JS047 | 5′-AACGCGCTACAGTCTGACGCTAAA-3′ | 32 |
| JS044 | 5′-biotin-GCACGTCAGGCACGGCGTCTGAAGATCAGTTGGGTGCA | 33 |
| | CGAGT-3′ | |
| JS040 | 5′-TGAAGATCAGTTGGGTGCACGAGT-3′ | 34 |
| JSO41 | 5′-AATAGTGTATGCGGCGACCGAGTT-3′ | 35 |
| JS042 | 5′-AACTCGGTCGCCGCATACACTATT-3′ | 36 |
| JS043 | 5′-TGTCAGAAGTAAGTTGGCCGCAGT-3′ | 37 |
| JS119 | 5′-GGCAAGGCCGGATCCAGAC-3′ | 38 |
| JS126 | 5′-GTCAGGTTCACCGATGGAGCG-3′ | 39 |
| JS140 | 5′-P-GTCCGCTCGAGACACCGAAAACGGTGTCTCGAGCGGACCA | 40 |
| | CTGACTGTATGATGAAGATGCTGACGAGGTCTGGATCCGGCCTTG | |
| | CCGCAGCTGGTAC-ddC-3′ | |
| JS141 | 5′-biotin-GCACGTCAGGCACGGCGTCGGTACCAGCTGCGGCAAGG | 41 |
| | CCGGATCCAGAC-3′ | |
| CPD oligomer | 5′-CTCGTCAGCATCTTCATCATACAGTCAGTG-3′ | 42 |

Preparation of Plasmid DNA.

Plasmid pCEP4 was purchased from Life Technologies. To obtain DNA sequences that were synthesized biologically with high accuracy, plasmids were produced in three E. coli strains: JM101, NM522 and Top10 for comparison. The pCEP4 plasmid was electroporated into E. coli and the bacteria were grown in medium for 26 population doublings (PDLs). Plasmids were isolated using the Maxi-Prep kit from Qiagen. Using the modified RMC assay, the error frequencies of the plasmids produced in JM101, NM522 and Top10 were measured as 3.1×10⁻⁶, 5.9×10⁻⁶ and 1.1×10⁻⁶, respectively. Since Top10 had the lowest mutation frequency, this strain was used as the host cell to optimize PDLs for the lowest possible mutation frequency of the plasmids. It was observed that bacteria with longer growing times (more population doublings) yielded plasmids with higher mutation frequencies. The mutation frequencies in plasmid pCEP4 grown in Top10 for 23 PDLs, 26 PDLs and 36 PDLs were 6.3×10⁻⁷, 1.3×10⁻⁶ and 2.8×10⁻⁶, respectively.

Preparation of Phage DNA.

M13mp18 RF I DNA (New England BioLab) was used to transform electro-competent cells of E. coli JM109 (F′) and infected plaques from a top agar lawn containing Xgal/IPTG and JM109 (F′) master stock were transferred to LB medium and used to JM109 (F′) bacteria in LB broth. After 6 h incubation with shaking at 37° C., the bacteria were collected by centrifugation and the supernatant containing M13 phage was saved as phage stock (about 10¹² pfu/ml) for future infection. To prepare M13mp18 ssDNA in large amounts, a small volume (2.5 ml) of plating bacteria (JM109 (F′) or CSH50 master stock) was infected by adding the M13 phage stock (0.5 ml) to the bacterial culture. After 5 min incubation at room temperature, the infected bacterial solution (3 ml) was transferred to and mixed with a large volume (250 ml) of pre-warmed LB medium (37° C.). This bacterial solution was incubated for 5 h, at 37° C., with vigorous shaking (250 rpm).

After incubation, the cells were collected (10,000×g), and the supernatant that contains M13 phage used for subsequent purification of the phage DNA. To precipitate the phage from the collected supernatant, 0.2 volume of chilled Buffer M1 (30% PEG 8000 and 3 M NaCl) was added to the phage solution, incubated it on ice for 1 h, and pelleted the phage particles by centrifugation at 10,000×g. The pellets were then resuspended in Buffer M2 (1% Triton X-100, 500 mM Guanidine-HCl and 10 mM MOPS, pH 6.5) and incubated in an 80° C. water-bath for 40 min to dissolve the phage envelopes. Finally, the phage lysates were applied to a Qiagene-tip-500 column for purification of phage ssDNA, following the manufacturer's protocol.

Construction of Oligonucleotide Probes.

Synthetic double-stranded oligomers (49 bp dsDNA)—Equal molar amounts of two complementary strands of chemically synthesized oligonucleotides (JS001 and JS002, Tables 1 & 2) were mixed together in DNA annealing buffer (10 mM Tris-HCl, pH 7.5, 50 mM NaCl and 1 mM EDTA) and boiled in a water bath for 3-5 min. After boiling, the DNA solution was kept in the water bath until it cooled to room temperature (23° C.).

TABLE 2

| DNA Probe | Components |
|---|---|
| Synthetic dsDNA | JS001 + JS002 |
| D-loop A | JS044 + pCEP4/AatII |
| Primer Extension | JS065 + M13mp18 ssDNA/AfeI-BaeGI |
| Bypass (G/running start) | JS124 + JS122 |
| Bypass (G/standing start) | JS125 + JS122 |
| Bypass (G = O/running start) | JS124 + G = O template |
| Bypass (G = O/standing start) | JS125 + G = O template |
| NER | CPD oligomer + JS140 + JS141 |

D-Loop.

The D-loop structure was formed by inserting a synthetic oligomer into a restriction fragment of plasmid DNA. DNA fragments derived from AatII digestion of the plasmid pCEP4 were used as templates (Tables 1 & 2). The oligomer contained a 3′ sequence homologous to the insertion site of the dsDNA (plasmid base 6272-6295, which is 8 base-pairs 5′ upstream of the target TCGA sequence, in the AatII-cleaved pCEP4 fragment of base 6040-8436), a 5′ sequence with a unique barcode for identification, and a biotin residue at the 5′-terminus to facilitate later isolation and enrichment of the probes from cellular extracts by streptavidin bead binding. The 5′-biotin tagged oligomers were mixed in 100 times excess molar concentration with the dsDNA fragments in DNA annealing buffer, boiled, and cooled gradually to room temperature (23° C.). The excessive amount of the synthetic oligomers increases the probability that the 3′ homologous sequence will pair with its complementary nucleotides of the dsDNA and displace the canonical opposite strand, forming a triplex, D-loop (displacement-loop) structure (Johnson & Jasin 2001; Li & Heyer 2008).

Primer Extension.

The template/substrate was a synthetic oligomer, containing a 3′ homologous sequence to the target template, a 5′ barcoded sequence and a 5′-biotin terminal cap, hybridized to a single-stranded DNA fragment derived from M13 phage (Tables 1 & 2). The linear M13mp18 ssDNA was formed, firstly, by annealing two synthetic oligonucleotides to form duplex DNA at restriction sites AfeI and BaeGI; and secondly, by restriction digestion. After restriction digestion, the linear ssDNA fragment was released from the closed circular form M13 ssDNA and annealed with the 5′-biotinylated oligomer to form a primer/template substrate for strand elongation by DNA polymerases.

Lesion Bypass.

The template DNA was synthesized by Midland and harbored a single residue of 8-oxo-dG (G=O) at a TaqI target site, T-C-G=G-A (Tables 1 & 2). The template was then hybridized with an equal molecular concentration of synthetic complementary primer with the 3′-terminal base pairing with the template base either right before (for standing start insertion) (Mendelman et al. 1989) or one base away (for running start insertion) from the G=O lesion.

T-T Dimer.

The DNA construct containing a cyclobutane pyrimidine dimer (CPD) was created by ligation of three synthetic oligonucleotides: a 5′-biotinylated oligomer, a 30 nt ssDNA containing a CPD dimer in the center of the sequence, and a fold-back oligonucleotide which forms a hairpin and also serves as a complementary strand for the other two oligomers to form a complete dsDNA construct (FIG. 1A). The 5′-termini of the hairpin oligonucleotides were chemically phosphorylated to facilitate ligation and the 3′-termini were blocked by addition of a dideoxycytosine (ddC) residue to prevent the DNA from exonuclease degradation. Since the CPD oligomers do not contain a 5′-phosphate, 5′-phosphorylation of the oligonucleotides was carried out by the T4 polynucleotide kinase in the presence of ATP. The three oligonucleotides (Tables 1 & 2) were hybridized together at equivalent molarities by boiling and re-annealing in a water bath. T4 DNA ligase was used to seal the nicks between the oligomers and complete the hairpin dsDNA construct.

Isolation of Nuclei.

Cell nuclei were isolated by treating the cells with hypotonic buffer; followed by gentle homogenization and subcellular separation by centrifugation. Cells were harvested in pellets and resuspended in hypotonic buffer (10 mM Hepes, pH 7.5, 2 mM MgCl₂, 25 mM KCl, 1 mM DTT, 1 mM PMSF and 1:200 (v/v) Protease Inhibitor cocktail III, CalBiochem). Depending on cell types, the cellular suspension was incubated on ice for different periods of time prior to homogenization using a Dounce Homogenizer with pestle B. Immediately after homogenization, 200 mM sucrose was added to the cell lysates and centrifugation was carried out to spin down the nuclei from other cellular debris. The nuclei are immediately ready for experiments or can be stored in −80° C. for later use.

Cell Culture, Transfection and Oligonucleotide Probe Retrieval.

Cell lines that have been used in this experiment include human embryonic kidney cell HEK293T, SV40-transformed normal human fibroblasts GM00637, SV40-transformed human XPA fibroblasts GM04429 (courtesy of Dr. Junko Oshima, University of Washington), human primary fibroblasts (Normal Human Dermal Fibroblasts, NHDF), normal human primary fibroblasts AG01440 and human XPA primary fibroblasts AG06971 (Coriell Institute). HEK293T, GM00637 and GM04429 were propagated in Dulbecco's Modification of Eagle's Medium (DMEM, Mediatech) supplemented with 10% fetal bovine serum (FBS), 100 U/ml penicillin G, 0.1 mg/ml streptomycin and 2 mM Lglutamine. NHDF, AG01440 and AG06971 were grown in the same medium except supplemented with 15% FBS. Cultures were maintained in a humidified incubator at 37° C. with 95% air and 5% CO₂; and transfection of cells with DNA probes was mediated either by calcium phosphate/DNA precipitate or by Lipofectamine 2000 (Life Technologies).

Transfection of cells with DNA probes was mediated by calcium phosphate/DNA precipitate. To retrieve the DNA probes from the transfected cells, the DNA extraction kit from Stratagene was used to isolate total DNA from the cells. As a biotin molecule had been added at the 5′-end of the probes, the DNA probes were purified by binding to streptavidin beads (Invitrogen). After several washes following the manufacturer's protocol, the DNA probes were released from the beads by incubation at 80° C. in deionized water.

Real-Time Quantitative PCR Analysis and the Modified RMC Assay.

The assay of Random Mutation Capture (RMC) was developed to detect mutations in genomic DNA. Briefly, it employs restriction enzyme digestion at a specific nucleotide sequence and subsequent real-time quantitative PCR (qPCR) to quantify the number of non-cleaved DNA molecules due to mutations within the restriction sequence. The RMC assay can detect non-canonical bases at a frequency of 1×10⁻⁸ per base. In the Oligonucleotide Probes Retrieval assay, a TaqI site (TCGA) was constructed in the target sequence. After retrieving the DNA probes from the transfected cells, the probes were digested 10 rounds with the TaqI restriction enzyme, and subjected the product to qPCR analysis. By using two primers flanking the TaqI site, the probes escaping TaqI cleavage due to DNA synthesis errors were amplified by PCR cycling (Ct_{(Test)}; Ct, cycling threshold). The amplified signals were quantified by normalization with PCR signals of a non-TaqI segment adjacent to the target site (Ct_{(control)}). The ratio of the un-cleaved DNA to the total molecules tested reveals the mutation frequency (MF) of the target TCGA sequence (MF=1/2(2^{ΔCt}×4), ΔCt=Ct_{(Control)}−Ct_{(Test)}). Products obtained from the bypass and nucleotide analog experiments were verified by sequencing and/or gel analysis.

Calibration of Nucleotide Excision Repair.

The hairpin construct of dsDNA containing a cyclobutane pyrimidine dimer (CPD) was used as an oligonucleotide probe for assaying the capacity of nucleotide excision repair (NER) in cells. This probe contains a CPD lesion in the middle of the dsDNA stretch, allowing the cellular NER protein complex to excise and remove a long patch of nucleotides flanking the lesion, synthesize a new patch of DNA to fill in the gap, and rejoin the strands together by ligation (FIG. 1A). If not repaired, CPD will prevent the polymerase from copying DNA across the lesion in PCR reactions. By using primers flanking the CPD site, the repaired probes can be quantified based on amplification of the restored DNA strand by qPCR. A single-stranded region 5′ of the probe was utilized as a unique forward primer sequence to specify the CPD bearing strand for PCR amplification with the reverse primer sequence located ahead of or behind the CPD. The amplified signal by qPCR from the 5′-forward primer with the 3′-reverse primer ahead of the lesion serves as a control for the total number of the retrieved probes (Ct_{control}), whereas amplification using the same 5′-forward primer but with the 3′-reverse primer behind the CPD site represents the number of the repaired probes (Ctₜₑₛₜ). Thus, the repair efficiency, defined as % repaired, can be calculated from the ratio of the number of the repaired probe over the number of total probes retrieved (% repaired=(2^{-ΔCt})×100, ΔCt=Ct_{control}−Ctₜₑₛₜ). To proceed with the assay, the cells to be examined for NER capacity were transfected with the biotin-tagged CPD probe. After a designated time of incubation that permits the probe to enter the cells and get repaired, the probes were retrieved by streptavidin bead capture either from the whole cell extract or from the nuclei for evaluation of repair efficiency in the cells.

Results

Retrieval of Oligonucleotides from Live Cells.

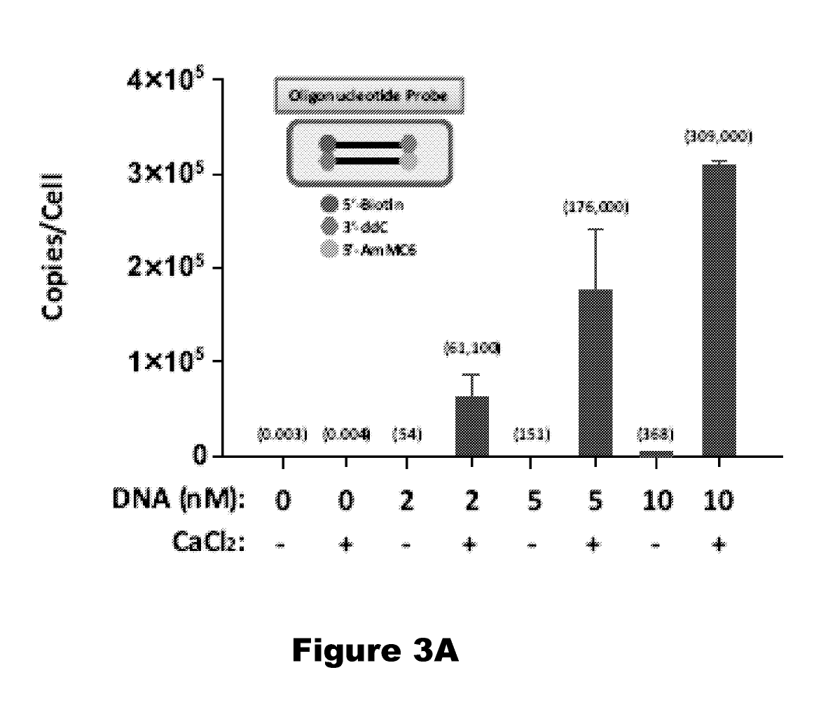

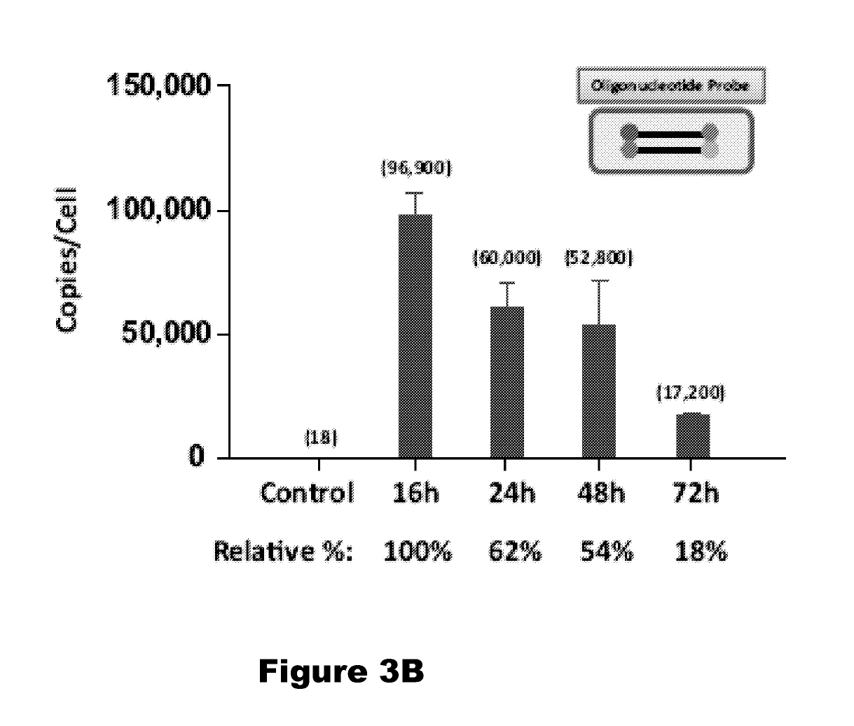

To establish an assay using DNA probes for in vivo assessment of the mutagenic potentials of human cells, the feasibility of transfecting and retrieving double stranded chemically synthesized deoxy-oligonucleotides was examined. Since the stability of the oligonucleotide probes in cells is critical to the assay, the 5′-end of one strand was protected with a biotin molecule (this also is important for later isolation by streptavidin binding), and the 5′-end of the complementary strand with 5′-AmMC6. The 3′-ends of both DNA strands were terminated with dideoxynucleotides to prevent exonucleolytic degradations (FIG. 2). An established human embryonic kidney cell line, HEK293T, was transfected with the protected oligonucleotide probe of different concentrations (0, 2, 5 and 10 nM) and after incubation at 37° C. for 16 h the probe was retrieved, followed by DNA extraction. Relative to an amplification of a control sequence on chromosome 17p13, it was demonstrated by qPCR that as many as 309,000 probe molecules per cell were able to be retrieved after incubation for 16 hrs (FIG. 3A), less than 0.1% of that was recovered in the absence of the transfection mediator calcium phosphate. The number of retrieved oligonucleotide probes was proportional to the amount of DNA used in the transfection. FIG. 3B demonstrates that when cells are transfected with 5 nM of the probe, more than 50,000 probes can be retrieved per cell, even after 48 hrs of incubation. Given the large number of probe molecules that can be introduced and their stability, it might be feasible to monitor DNA synthetic reactions, including the incorporation of noncomplementary nucleotides, in small numbers of cells.

Base Accuracy of Synthetic Oligonucleotides, Bacterial Plasmids and Bacteriophage DNAs.

In order to measure the accuracy of DNA transactions, the template DNA used in constructing OPRA probes must be of exceptional purity with essentially no nucleotide variation at each position. The modified RMC assay was used to measure the homogeneity of oligonucleotides obtained from a variety of commercial sources. Invariably the frequency of incorrect bases at a target TCGA site in synthetic oligonucleotides using the RMC assay was greater than 1.0×10⁻³ (Table 3). To derive template DNA with a lower inherent mutation frequency, the bacterial plasmid pCEP4 was purified from cells grown for a limited number of cell divisions. Under optimal growing conditions, double stranded plasmids were obtained having an error frequency of ˜1.0×10⁻⁶ per base (see Materials). Single-stranded DNA was isolated from the bacteriophage M13mp18 grown in E. coli. The base error frequency of bacteriophage ssDNA and dsDNA is 1.1×10⁻⁶ and 2.8×10⁻⁷ per base, respectively (Table 3). It should be noted that TaqI also efficiently cleaves TCGA sequences in single-stranded DNA in the modified RMC assay. Thus, using biologically derived DNA sequences, probe molecules can be constructed that are greater than 1,000 times more accurate than synthetic oligonucleotides.

TABLE 3

| | | Retrieved | | |
|---|---|---|---|---|
| | | Oligonucleotides | DNA Error Frequency (per base)* | |
| DNA Probe | Structure | (copies per cell) | Before Transfection | After Transfection |
| Synthetic Oligomers (49 bp dsDNA) | 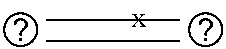 | 309,000 ± 4,850 | (1.2 ± 0.1) × 10⁻³ | NA |
| Primer/Synthetic ssDNA Template (49 nt) | 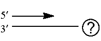 | 103,000 ± 2,980 | NA | NA |
| Elongated Primer | 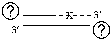 | 5,500 ± 396 | NA | NA |
| Plasmid Fragment (2.4 kb dsDNA) | 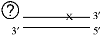 | 21,000 ± 923 | (6.6 ± 0.6) × 10⁻⁶ | (2.0 ± 0.8) × 10⁻⁵ |
| Primer/Plasmid dsDNA Template (D-loop) | 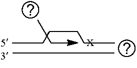 | 20,900 ± 2,050 | (8.2 ± 0.4) × 10⁻⁶ | NA |
| Elongated Primer | 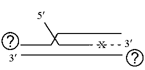 | 244 ± 47 | NA | (4.6 ± 0.1) × 10⁻⁵ |
| M13mp18 Phage (dsDNA) | 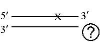 | NA | (2.8 ± 0.7) × 10⁻⁷ | NA |
| Primer/M13 ssDNA Template (949 nt) | 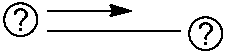 | 4,630 ± 205 | (1.1 ± 0.1) × 10⁻⁶ | NA |
| Elongated Primer | 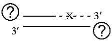 | 313 ± 25 | NA | (4.6 ± 0.7) × 10⁻⁵ |
| G═O Bypass Primer/Template (120 nt) | 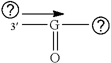 | 218,000 ± 44,600 | (4.4 ± 1.0) × 10⁻³ | NA |
| Elongated Primer bypassed G═O | 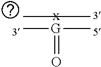 | 106 ± 9 | NA | (7.0 ± 2.0) × 10⁻² |

*Error frequencies were determined at the TCGA site “x”, oligonucleotide probes were transfected into HEK293T cells, and data are presented as mean ±5.0 from two independent experiments.
NA; Not Applicable

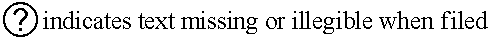

The Fidelity of Copying a Linear DNA Template In Vivo.

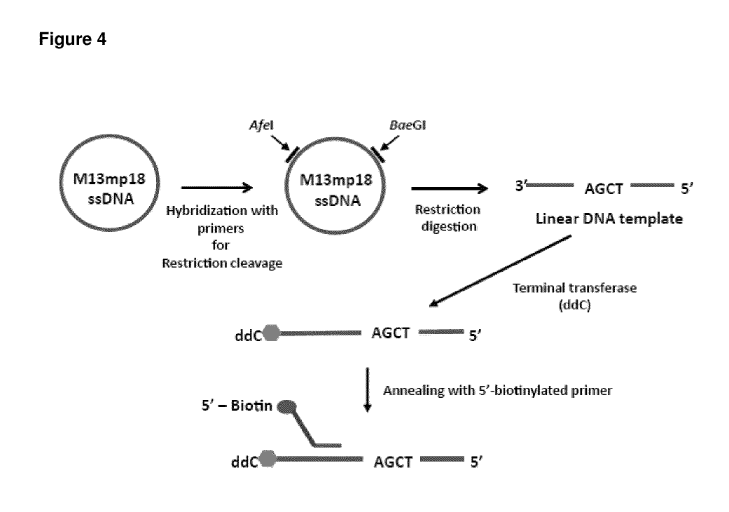

The copying of a single-stranded DNA template is central to DNA replication and a variety of other DNA transactions in cells. To assess the fidelity of de novo DNA synthesis, a DNA template for primer extension was derived from single-stranded M13mp18 bacteriophage DNA; the base accuracy of this ssDNA prior to transfection is 1.1×10⁻⁶ (Table 3). After annealing with primers at two specific restriction locations, the circular DNA was digested with two designated restriction enzymes to produce a single 7 stranded DNA fragment of a defined length. A synthetic primer was then hybridized onto a template with a 5′-biotin tag and a unique 5′-barcode sequence. The 3′-segment of the primer is complementary to the template and forms a partial duplex that can be extended by cellular DNA polymerases after transfection into cells (FIG. 4).

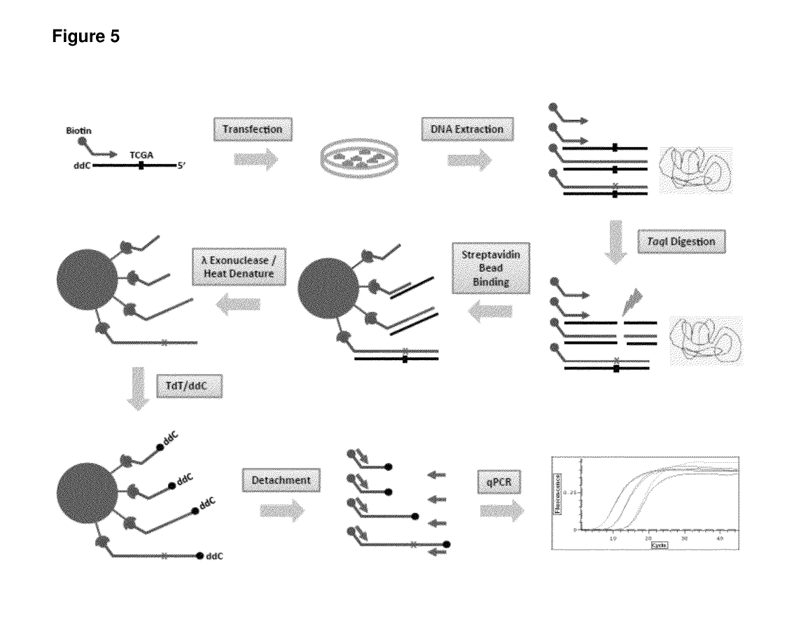

HEK293T cells (1×10⁶) were grown in a 60 mm dish and transfected using calcium phosphate with 0.1 nM of the template/primer complex. The transfected cells were maintained in a 37° C. incubator supplied with 5% CO₂. After 24 hours, cells were washed extensively and the template/primers were isolated from total DNA by complexing with streptavidin beads. After digestion with TaqI restriction enzyme, the mutation frequency was determined using the modified RMC assay (FIG. 5). About 5,000 copies of the template/primer probe were introduced into each cell; a total of 5×10⁹ probe molecules were retrieved and analyzed in each experiment. Of the 5,000 probes in each cell, 300 were elongated (6%) and the mutation frequency of single-base substitutions at the target TaqI site in the elongated strand was 4.6×10⁻⁵ per base (FIG. 6a); greater than tenfold above that of the initial template. Following the same procedure, the fidelity of primer extension was examined in primary human fibroblasts. The fibroblast cells (6×10⁶) were transfected with 0.1 nM of the primer/template probe and incubated at 37° C. for 24 h before DNA extraction. About 50,000 probes were retrieved from each cell and discovered that approximately 300 of them were elongated (0.6%). This result suggests that DNA synthesis in the primary fibroblasts is 10 times less active than that in the transformed kidney cells. With respect to the copying fidelity, it was found that the mutation frequency of the elongated DNA in the primary cells, at 9.4×10⁻⁶ per base (FIG. 6B), was 5 times lower than that in the transformed cells (FIG. 6A).

Mutagenicity of Nucleoside Analogs in Human Cells.

OPRA can also be used to measure the mutagenicity of nucleoside analogs that mispair at high frequencies (FIG. 7A). Human embryonic kidney cells 293T (8×10⁵) were cultured in 3.3 mM of 2-aminopurine (2-AP) or 2′-deoxyinosine (dl) for 20 hours. The cells were transfected with the primer extension probes in the absence of nucleoside analogs. After four-hour incubation, nucleoside analogs were added back to the cultures and the incubation continued for another 12 hours. DNA was extracted from the cells and used for subsequent mutational analyses. As illustrated in FIG. 7A, the 5′-biotin tagged primer could be elongated in cells in the presence of the triphosphate compounds of 2-AP or dl. The elongated DNA containing the mutagenic nucleotide analogs was isolated and enriched using magnetic beads conjugated to streptavidin. The template DNA annealed with the elongated strand was hydrolyzed by λ-exonuclease to generate the newly elongated DNA in single-stranded form. The newly elongated ssDNA was then copied by Taw polymerase using a single complementary primer. This copied strand contains the signature mutations derived from bases impairing with nucleotide analogs incorporated in vivo during DNA synthesis. The copied strand was then PCR amplified and its mutation frequency quantified using the modified RMC assay. In comparison with the untreated cells, cells treated with 2-AP exhibit a 2-fold increase of mutation frequency per base at the TCGA target site, while cells treated with dl show a 3-fold increase (FIG. 7B).

A D-Loop Substrate for Primer Extension In Vivo.

To simulate more complex DNA transactions, a DNA primer/template substrate was constructed to mimic a pseudo-D-loop structure. This represents an intermediate of homologous recombination (27, 28) (FIG. 8A) where a DNA polymerase must synthesize DNA by elongating the primer along the complementary strand while displacing the other strand of the template. The template DNA was an enzyme-restricted fragment of the plasmid pCEP4, which had a base error frequency of 6.6×10⁻⁶ per base (Table 3), while the primer was a chemically synthesized oligonucleotide with a 3′ sequence complementary to the template. The 5′-terminus of the primer carried a biotin residue used for purification and a unique barcode sequence that is not complementary to the template. This permitted selective PCR-amplification of the elongated primer. The substrate was constructed by annealing the barcoded, 5′-biotinylated primer and the double-stranded DNA fragment to form a pseudo-D-loop structure, simulating the 3′ strand invasion that occurs during double-strand-break-induced homologous recombination (Jasin & Johnson 2001; Li & Heyer 2008) (FIG. 8A). The D-loop substrate (0.1 nM) was transfected into HEK293T cells (1×10⁶ cells per dish). After 24 h incubation, the probe was retrieved, and the transfection efficiency, extent of primer extension, and mutation frequency of the newly-synthesized DNA were measured by the modified RMC assay. Approximately 20,000 copies of the D-loop substrate were introduced into each cell, of which, 200 copies were elongated past the TaqI site. The mutation frequency at the target TaqI site of the newly synthesized DNA was found to be 4.6×10⁻⁵ per base; while that of the template dsDNA not annealed to the primer was 2.0×10⁻⁵ per base, revealing a 2-fold increase of mutations in the primer extension reaction (FIG. 8B).

Nucleotide Excision Repair In Vivo.

Environmental agents can introduce bulky lesions in DNA that disrupt progression of DNA replication, for example, benzo(a)pyrene [B(a)P] diolepoxide (BPDE) adducts introduced by smoking (Rechkoblit et al. 2002; Hecht 1999) or cyclobutane pyrimidine dimer (CPD) generated by UV-radiation (Hanawalt et al. 2003). These lesions are subject to removal and re-synthesis by nucleotide excision repair; the patch of nucleotides (˜30 nt) encompassing the damaged residue is first excised, DNA is then synthesized to fill in the resulting gap, and finally, the DNA strands are ligated to complete the process. If not repaired, these bulky DNA adducts and intra-strand crosslinks block the progression of DNA polymerases during DNA replication and stall the replication fork, leading to checkpoint activation and the induction of error-prone repair.

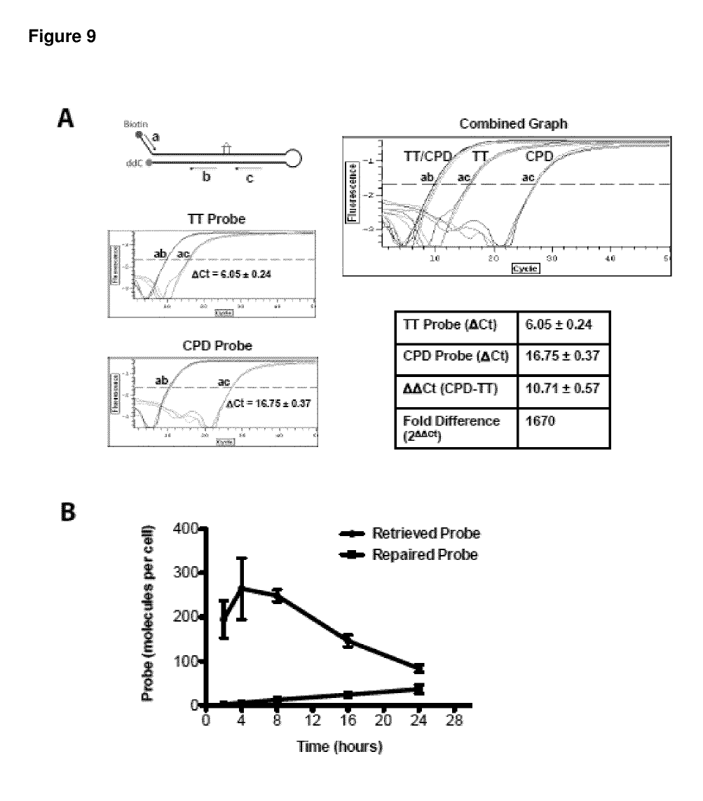

To measure the capacity of NER in vivo, an oligonucleotide probe was constructed harboring a site-specific cyclobutane pyrimidine dimer (CPD), the gold standard for UV induced DNA damage, in a hairpin duplex structure (FIG. 1A). The double-stranded region is 77-nt long and the CPD is located in the middle of the duplex DNA, providing sufficient length for excision by NER. The CPD adduct blocks the activity of Taq DNA polymerase during amplification by PCR (FIG. 9A) as tested using the primers flanking the CPD indicated in FIG. 1A. On the 5′-end of the probe, a terminal biotin residue is positioned for retrieval of the probe by streptavidin bead binding; and a single-stranded DNA region with a unique sequence specifies the 5′-forward primer used for qPCR amplification (FIG. 1A). The 3′-end was capped with a dideoxycytidine residue to prevent 3′ degradation by non-specific exonuclease activities.

5×10⁵ HEK293T cells were transfected with 0.02 nM of the CPD probe mediated by calcium phosphate. The CPD probe used for transfection was prepared by ligation as described previously, and to maximize yield was not further purified. After incubation, the cells were harvested at increasing times up to 24 h. Whole-cell DNA was extracted and the probes were isolated by streptavidin bead capture. The repaired probes, which unlike the input CPD-containing probes, are amplifiable and detected by qPCR. Approximately 200 probes per cell were retrieved at each time point (FIG. 9B); the number of repaired probes steadily increased in a time-dependent manner (FIG. 6B). NER activities were detected as early as 2 h after transfection. At 16 h, about 20% of the retrieved CPD probes per cell were restored to their canonical, qPCR-amplifiable sequence; at 24 h, it was 40%.

To validate that this assay measures the activities of NER, SV40-immortalized human fibroblast cell lines derived from a normal individual (GM00637) were compared with those from a xeroderma pigmentosum (XP) patient with defective XPA alleles (GM04429). After 48 h incubation, nuclei were isolated and CPD probes were retrieved from purified nuclei for quantification. As shown in FIG. 1C, the XPA cells are about 80% less efficient in repairing thymine dimers than normal cells, verifying that the assay is capable of detecting the NER deficiency in XP cells. Using primary human fibroblasts AG01440, derived from a healthy individual, and AG06971, obtained from an individual with XPA deficiency, it was observed that after 48 h incubation, the normal fibroblasts repaired 0.7% of the retrieved probes, compared to 0.4% for the XPA cells. After 96 h, the normal fibroblasts had repaired 13% of the input CPD probe, whereas the XPA fibroblasts had repaired only 1.5% of the probes −90% lesser efficiency than that of the normal cells (FIG. 1D). This result confirms the utility of this assay in assessing the DNA repair capacity of various cell types including human primary cell cultures.

DNA End-Joining in Human Cells.

A major mechanism for recombination in human cells is end-joining of DNA fragments, which can introduce errors into DNA. Thus, a fragment of plasmid pCEP4 was employed to mimic the intermediate of this mechanism. As illustrated in FIG. 10A, pCEP4 DNA was cleaved with SalI (G/TCGAC), yielding the 5′-overhang sequence TCGA. The 8.9 kb linearized DNA fragments were gel purified and transfected into human HEK293T cells. Intra-molecular end-joining of the SalI termini in the transfected cells would yield a PCR-amplifiable product using primers flanking the cleavage site. Relative to a control sequence on chromosome 17p13, PCR amplification of an internal region of the 8.9 kb DNA fragment was used as a copy number control. Of the 3,000 molecules of the DNA fragment introduced into each cell, about 700 end-ligated termini were detected, indicating active end-joining reactions in these cells. In addition the sequences of 12 such end-joined molecules were determined by sequencing qPCR products derived from single molecules (FIG. 10B). The results of this single molecule sequencing are presented in FIG. 10B and reveal diverse mechanisms used by cells to rejoin DNA double strand breaks.

At least four hypothetical mechanisms can be deduced from the observed end-joining products (see details in the legend of FIG. 10B). 1. Direct cohesive end ligation (3 of 12; A3, C2 and C8), which results in restoration of the wild-type SalI sequence GTCGAC. 2. Filled-in blunt end ligation (3 of 12; A10, A12 and C10), which involves DNA synthesis on both termini and results in a repeat of TCGA in the SalI site. 3. Partial filled-in end ligation (2 of 12; D10 and D12), which may involve both Flap-endonuclease activities and partial filled-in DNA synthesis on one terminus, resulting in insertion of either TC or GA of the TCGA sequence in the SalI site. 4. Recessed blunt end ligation (4 of 12; B2, B4, B11 and C5), which results in deletion of the SalI site.

Measuring Lesion-Bypass Efficiency In Vivo Using Oligonucleotide Probes.

It is estimated that the genome of each human cell is subjected to approximately 50,000 DNA damaging events per day (Paz-Elizur et al. 2003). Some of these base lesions are not excised prior to DNA replication and their bypass during DNA replication is essential for cells to avoid the collapse of stalled replication forks and thereby maintain genomic integrity.

To study the frequency and fidelity of lesion bypass, OPRA was employed to introduce synthetic primer/template oligonucleotides containing modified bases. Specifically, primer/template probes carrying a site-specific 8-oxo-dG (G=O) were introduced in the template strand into HEK293T cells by transient transfection mediated by calcium phosphate. A probe containing the canonical G residue at the target site served as a control. The 5′ of the bypassing primer contains a unique barcoded sequence to be used as a primer for PCR amplifications that specify the copy number of the intact probes and serve as an internal control. The 3′ terminus of the primer was positioned either one nucleotide upstream from the target G=O residue of the template referred to as “running start” DNA synthesis, or adjacent to the G=O site for “standing start” synthesis (Bertram et al. 2010). Approximately 200,000 probes were taken up per cell after a 17-hour incubation; while 1% of the G probes were elongated, only ˜0.1% of the G=O probes were extended past the lesion (FIG. 11A). The efficiency of DNA elongation past the G=O lesion relative to the canonical TCGA site in the same sequence context was approximately 10%. The efficiency of DNA elongation past the OG lesion relative to the canonical TCGA site in the same sequence context was approximately 10%. Compared to the plasmid-based experiments of Avkin and Livneh (Avkin & Livneh 2002), for example, where a small number of template molecules enter each cell, high concentrations of template per cell may be introduced. Since the repair capacity of the cell is limiting, under these conditions only a subset of templates is bypassed. Bypass synthesis was highly error prone. There was no significant difference in the percentage of probes extended whether or not a running start or a standing start primer was used. Furthermore, this bypassing synthesis was highly error prone. Measured by the modified RMC assay, error frequencies of bypassing the G=O lesion were about 7% per base for both of the running start and the standing start primers (FIG. 11B). To examine the spectrum of the bypass errors, single molecules of the elongated standing start primers were cloned and sequenced. Of 75 sequences obtained from the elongated control G probe, no mutations were found. However, of 35 G=O bypassed probe sequences, three C to A transversions were observed—the predicted result of G=O mispairing with A. Thus from direct sequencing of the bypass products, the mutation frequency of bypassing G=O is 8.6% (3 of 35), similar to the initial calculation (7%, modified RMC assay).

### REFERENCES

The references, patents and published patent applications listed below, and all references cited in the specification above are hereby incorporated by reference in their entirety, as if fully set forth herein.

### REFERENCES

Albertini, R. J., Nicklas, J. A., O'Neill, J. P. & Robinson, S. H. In vivo somatic mutations in humans: measurement and analysis. Ann. Rev. Genet. 24, 305-326 (1990).
Albertson, T. M. et al. DNA polymerase epsilon and delta proofreading suppress discrete mutator and cancer phenotypes in mice. Proc. Natl. Acad. Sci. U.S.A. 106, 17101-17104 (2009).
Arana, M. E. & Kunkel, T. A. Mutator phenotypes due to DNA replication infidelity. Semi. Cancer Biol. 20, 304-311 (2010).
Avkin, S. & Livneh, Z. Efficiency, specificity and DNA polymerase-dependence of translesion replication across the oxidative DNA lesion 8-oxoguanine in human cells. Mut. Res. 510, 81-90 (2002).
Avkin, S. et al. Quantitative analysis of translesion DNA synthesis across a benzo[a]pyrene-guanine adduct in mammalian cells. J. Biol. Chem. 279, 53298-53305 (2004).
Barnes D E, Lindahl T (2004) Repair and genetic consequences of endogenous DNA base damage in mammalian cells. Ann Rev Genet. 38:445-476.
Bertram J. G., Oertell K., Petruska J. & Goodman M. F. DNA polymerase fidelity: comparing direct competition of right and wrong dNTP substrates with steady state and pre-steady state kinetics. Biochemistry 49, 20-28 (2010).
Bielas, J. H. & Loeb, L. A. Quantification of random genomic mutations. Nat. Methods 2, 285-290 (2005).
Bourre, F., Renault, G. & Gentil, A. From simian virus 40 to transient shuttle vectors in mutagenesis studies. Mut. Res. 220, 107-113 (1989).
Chapados, B. R. et al. Structural basis for FEN-1 substrate specificity and PCNA-mediated activation in DNA replication and repair. Cell, 116, 39-50 (2004).
Chen, R., Eshleman, J. R., Brodsky, R. A. & Medof, M. E. Glycophosphatidylinositol-anchored protein deficiency as a marker of mutator phenotypes in cancer. Cancer Res. 61, 654-658 (2001).
Cleaver J E (2005) Cancer in xeroderma pigmentosum and related disorders of DNA repair. Nat Rev Cancer 5:564-573.
Cleaver J E, Lam E T, Revet I (2009) Disorders of nucleotide excision repair: the genetic and molecular basis of heterogeneity. Nat Rev Genet. 10:756-768.
Cleaver J E, Thomas G H (1981) Measurement of unscheduled synthesis by autoradiography. DNA repair, A laboratory Manual of Research Procedures 1:277-287 (Marcel Dekker, New York).
Collins A R (2004) The comet assay for DNA damage and repair: principles, applications, and limitations. Mol Biotech 26:249-261.
D'Andrea A D, Grompe M (2003) The Fanconi anaemia/BRCA pathway. Nat Rev Cancer 3:23-34.
Feng Z, Hu W, Tang M S (2004) Trans-4-hydroxy-2-nonenal inhibits nucleotide excision repair in human cells: a possible mechanism for lipid peroxidation induced carcinogenesis. Proc Natl Acad Sci USA 101:8598-8602.
Friedberg E C, et al. (2005) DNA Repair and Mutagenesis (ASM Press, Washington D.C.) Second Ed.
Ganesan, A. K., Hunt, J. & Hanawalt, P. C. Expression and nucleotide excision repair of a UV-irradiated reporter gene in unirradiated human cells. Mut. Res. 433, 117-126 (1999).
Hanahan, D. & Weinberg, R. A. Hallmarks of cancer: the next generation. Cell 144, 646-674 (2011).
Hanawalt P C, Ford J M, Lloyd D R (2003) Functional characterization of global genomic DNA repair and its implications for cancer. Mut Res 544:107-114.
Hecht S S (1999) Tobacco smoke carcinogens and lung cancer. J Natl Cancer Inst 91:1194-1210.
Helleday T, Petermann E, Lundin C, Hodgson B, Sharma R A (2008) DNA repair pathways as targets for cancer therapy. Nat Rev Cancer 8:193-204.
Hu J J, et al. (2004) Deficient nucleotide excision repair capacity enhances human prostate cancer risk. Cancer Res 64:1197-1201.]
Jasin, M. & Johnson, R. D. Double-strand-break-induced homologous recombination in mammalian cells. Biochem. Soc. Trans. 29, 196-201 (2001).
Johnson R D, Jasin M (2001) Double-strand-break-induced homologous recombination in mammalian cells. Biochem Sci Trans 29:196-201.
Kamath-Loeb A S, Shen J C, Schmitt M W, Loeb L A (2012) The Werner syndrome exonuclease facilitates DNA degradation and high fidelity DNA polymerization by human DNA polymerase delta. J Biol Chem 287:12480-12490.
Kim M Y, Wogan G N (2006) Mutagenesis of the supF gene of pSP189 replicating in AD293 cells cocultivated with activated macrophages: roles of nitric oxide and reactive oxygen species. Chem Res Toxicol 19:1483-1491.
Latimer J J, et al. (2010) Nucleotide excision repair deficiency is intrinsic in sporadic stage I breast cancer. Proc Natl Acad Sci USA 107:21725-21730.
Lee D H, Pfeifer G P (2008) Translesion synthesis of 7,8-dihydro-8-oxo-2′-deoxyguanosine by DNA polymerase eta in vivo. Mut Res 641:19-26.
Li, X. & Heyer, W. D. Homologous recombination in DNA repair and DNA damage tolerance. Cell Res. 18, 99-113 (2008).
Lindahl T, Wood RD (1999) Quality control by DNA repair. Science 286:1897-1905.
Liu Y, et al. (2010) Interactions of human mismatch repair proteins MutSalpha and MutLalpha with proteins of the ATR-Chk1 pathway. J Biol Chem 285:5974-5982.
Loeb, L. A. Human cancers express mutator phenotypes: origin, consequences and targeting. Nat. Rev. Cancer 11, 450-457 (2011).
McCulloch S D, Kunkel T A (2008) The fidelity of DNA synthesis by eukaryotic replicative and translesion synthesis polymerases. Cell Res 18:148-161.
McKinnon P J (2009) DNA repair deficiency and neurological disease. Nat Rev Neurosci 10:100-112.
Mendelman L V, Boosalis M S, Petruska J, Goodman MF (1989) Nearest neighbor influences on DNA polymerase insertion fidelity. J Biol Chem 264:14415-14423.
Miller, J. H., Lebkowski, J. S., Greisen, K. S. & Calos, M. P. Specificity of mutations in transfected DNA by mammalian cells. EMBO J. 3, 3117-3121 (1984).
Newman J C, Bailey A D, Weiner A M (2006) Cockayne syndrome group B protein (CSB) plays a general role in chromatin maintenance and remodeling. Proc Natl Acad Sci USA 103:9613-9618.
Patel, P. H., Kawate, H., Adman, E., Ashbach, M. & Loeb L. A. A single highly mutable catalytic site amino acid is critical for DNA polymerase fidelity. J. Biol. Chem. 276, 5044-5051 (2001).
Paz-Elizur, T. et al. DNA repair activity for oxidative damage and risk of lung cancer. J. Natl. Cancer Inst. 95, 1312-1319 (2003).
Rechkoblit O, et al. (2002) Trans-lesion synthesis past bulky benzo[a]pyrenediol epoxide N2-dG and N6 dA lesions catalyzed by DNA bypass polymerases. J Biol Chem 277:30488-30494.
Schmitt, M. W. et al. Active site mutations in mammalian DNA polymerase delta alter accuracy and replication fork progression. J. Biol. Chem. 285, 32264-32272 (2010).
Silverman L, Glick D (1969) Measurement of protein concentration by quantitative electron microscopy. J Biol Chem 40:773-778.
Spivak G (2004) The many faces of Cockayne syndrome. Proc Natl Acad Sci USA 101:15273-15274.
Spivak, G. & Hanawalt, P. C. Host cell reactivation of plasmids containing oxidative DNA lesions is defective in Cockayne syndrome but normal in UV sensitive syndrome fibroblasts. DNA Repair 5, 13-22 (2006).
Wang H T, Choi B, Tang M S (2010) Melanocytes are deficient in repair of oxidative DNA damage and UV-induced photoproducts. Proc Natl Acad Sci USA 107:12180-12185.
Wood R D, Robins P, Lindahl T (1988) Complementation of the xeroderma pigmentosum DNA repair defect in cell-free extracts. Cell 53:97-106.
Wu X, et al. (2005) Cell cycle checkpoints, DNA damage/repair, and lung cancer risk. Cancer Res 65:349-357.
Ziv, O., Geacintov, N., Nakajima, S., Yasui, A. & Livneh, Z. DNA polymerase cooperates with polymerase and in translesion DNA synthesis across pyrimidine photodimers in cells from XPV patients. Proc. Natl. Acad. Sci. U.S.A. 106, 11552-11557 (2009).

What is claimed is:

1. A method of measuring at least one DNA integrity transaction in a cell, comprising:
transfecting the cell with a plurality of oligonucleotide probes, the probes being blocked at the 5′ end, the 3′ end, or both, and comprising a tagged residue;
retrieving the plurality of oligonucleotide probes by targeting the tagged residue; and
quantifying the DNA integrity transaction, wherein the DNA integrity transaction involves one or more steps or processes that are responsible for restoring the integrity of DNA in a cell.

2. The method of claim 1, wherein the plurality of oligonucleotides is synthetic or are derived from natural DNA that mimic intermediates of the DNA integrity transaction.

3. The method of claim 1, wherein the at least one DNA integrity transaction is selected from DNA repair, DNA mutation, and DNA replication fidelity.

4. The method of claim 3, wherein the DNA repair is nucleotide excision repair (NER), base excision repair (BER), mismatch repair (MMR), homologous recombination (HR), and non-homologous end joining (NHEJ).

5. The method of claim 3, wherein DNA replication fidelity is replicative DNA synthesis fidelity, repair DNA synthesis fidelity, or translesion DNA synthesis fidelity.

6. The method of claim 1, wherein the at least one DNA integrity transaction comprises DNA repair, DNA mutation, and DNA replication fidelity measured during a single assay.

7. The method of claim 1, wherein the tagged residue comprises a sequence that is specific to DNA repair, DNA mutation, or DNA replication fidelity.

8. The method of claim 1, wherein the cells are human cells.

9. The method of claim 1, wherein the plurality of oligonucleotide probes comprises approximately between four and 200,000 oligonucleotide probes.

10. The method of claim 1, wherein the method is part of an automatic, high-throughput screening platform.

11. A method of determining a personalized cancer treatment for a cancer patient comprising
measuring a repair capacity of a cancer cell derived from the cancer patient in response to treatment with an effective amount of one or more chemotherapeutic agents;
determining a responsiveness of the cancer cell to the one or more chemotherapeutic agent; and
selecting one or more chemotherapeutic agents that are responsive in the cancer cell for the personalized cancer treatment, wherein:
a. the cancer cell is responsive to the one or more chemotherapeutic agents when the repair capacity is low; and
b. the cancer cell is not responsive to the one or more chemotherapeutic agents when the repair capacity is high.

12. The method of claim 11, wherein the repair capacity is measured by a method comprising
transfecting the cell with a plurality of oligonucleotide probes, the probes being blocked at the 5′ end, the 3′ end, or both, and comprising a tagged residue;
retrieving the plurality of oligonucleotide probes by targeting the tagged residue; and
quantifying the DNA repair capacity.

13. The method of claim 12, wherein the one or more chemotherapeutic agents can be incorporated into DNA and are selected from nucleoside analogs, nucleotide analogs, nucleobase analogs, antimetabolites, purine analogues, antifolates, pyrimidine analogues.

14. The method of claim 13, wherein quantifying the DNA repair capacity is accomplished by measuring the incorporation of the chemotherapeutic agent into the oligonucleotide probe.

15. The method of claim 13, further comprising identifying incorporation of one or more metabolized nucleoside analogs in one or more of the plurality of oligonucleotide probes by mass spectrometry.

16. The method of claim 13, wherein the oligonucleotide probes are covalently linked to the one or more chemotherapeutic agents and quantifying the DNA repair capacity is accomplished by the ability of the cancer cell to excise the one or more chemotherapeutic agent.

17. The method of claim 12, wherein the quantification of the DNA repair capacity is accomplished by real-time quantitative PCR.

18. A method of determining the ability of a putative mutagen to induce a DNA mutation in a human or mammalian cell comprising:
administering an effective amount of the mutagen to the cell;
measuring a DNA mutation frequency or DNA repair rate in the cell; and
determining that:
a. the mutagen is effective at inducing a mutation when the DNA repair rate is low or the DNA mutation rate is high; and
b. the mutagen is not effective at inducing a mutation when the DNA repair rate is high or the DNA mutation rate is low.

19. The method of claim 18, wherein the cell is part of a population of cells taken from a plurality of similarly situated subjects.

20. The method of claim 18, wherein the DNA repair rate or mutation rate is measured by a method comprising
transfecting the cell with a plurality of oligonucleotide probes, the probes being blocked at both the 5′ and 3′ ends and comprising a tagged residue;
retrieving the plurality of oligonucleotide probes by targeting the tagged residue; and
quantifying the DNA repair rate.

21. The method of claim 18, wherein the mutagens are nucleoside analogs or DNA damaging agents such as alkylating or crosslinking agents.

22. The method of claim 20, wherein a pre-mutagenic load incurred by administration of the mutagen is quantified by mass spectrometry.

23. A method for providing an index for classifying tumor types comprising:
measuring fidelity of DNA synthesis in a plurality of cancer cells derived from a plurality of tumor types; and
providing an index by establishing a reference error rate or range of error rates for each tumor type;
wherein the fidelity of DNA synthesis is measured by a method comprising
transfecting the cancer cells from each tumor type with a plurality of oligonucleotide probes derived from natural DNA, wherein the probes include an unblocked 3′ end that is elongated in the cell and comprise a tagged residue;
retrieving the plurality of oligonucleotide probes by targeting the tagged residue; and
quantifying the DNA repair capacity.
